SEQUENCE LISTING (2 sequences)

SEQ ID NO: 1 - DNA, 20 bp, Artificial Sequence
  Synthetic Construct
tgtgtgtgtgtgtgtgtgtg

SEQ ID NO: 2 - DNA, 20 bp, Artificial Sequence
  Synthetic Construct
cacacacacacacacacaca

(19) United States
(12) Patent Application Publication
(10) Pub. No.: US 2010/0152281 A1
(43) Pub. Date: Jun. 17, 2010

(54) METHODS OF TREATING SYSTEMIC LUPUS ERYTHEMATOSUS IN INDIVIDUALS HAVING SIGNIFICANTLY IMPAIRED RENAL FUNCTION

(76) Inventors: Matthew D. LINNIK, Solana Beach, CA (US); Bonnie HEPBURN, Escondido, CA (US); Robert G. BAGIN, Julian, CA (US)

Correspondence Address: MORRISON & FOERSTER LLP, 755 PAGE MILL RD, PALO ALTO, CA, 94304-1018, US

(21) Appl. No.: 12/263,239

(22) Filed: Oct. 31, 2008

Related U.S. Application Data
(63) Continuation of application No. 10/219,238, filed on Aug. 13, 2002, now abandoned.
(60) Provisional application No. 60/311,858, filed on Aug. 13, 2001.
(60) Provisional application No. 60/314,281, filed on Aug. 22, 2001.

(51) Int. Cl. A61K 31/713; A61P 37/02; A61P 13/12

(52) U.S. Cl. 514/44 R

(57) ABSTRACT

The invention provides methods treating lupus nephritis based in individuals with significantly impaired renal function, and methods of selecting individuals for treatment based on significantly impaired renal function. The treatment entails administration of a conjugate comprising a non-immunogenic valency platform molecule and at least two double stranded DNA epitopes, such as DNA molecules, which bind to anti-DNA antibodies from the patient. The invention also provides methods of identifying individuals suitable for treatment for lupus, based on assessing renal function to identify those individuals with significant impairment of renal function.

### CROSS-REFERENCE TO RELATED APPLICATIONS

This application claims priority under 35 U.S.C. §119(e) to U.S. Provisional Patent Application Ser. No. 60/311,858, filed Aug. 13, 2001, and to U.S. Provisional Patent Application Ser. No. 60/314,281, filed Aug. 22, 2001, each of which is hereby incorporated by reference in its entirety.

### TECHNICAL FIELD

This invention relates to the field of antibody-mediated pathologies such as lupus. More particularly, the invention relates to methods of treating individuals with systemic lupus erythematosis having significantly impaired renal function.

### BACKGROUND ART

Systemic lupus erythematosis (SLE) is an autoimmune disease characterized by the production of antibodies to a number of nuclear antigens, including double-stranded DNA (dsDNA). Autoantibodies that react with DNA are believed to play a role in the pathology of SLE and are closely associated with lupus nephritis. See, for example, Morimoto et al. (1982) J. Immunol. 139:1960-1965; Foster et al. (1993) Lab. Invest. 69:494-507; ter Borg et al. (1990) Arthritis Rheum. 33:634-643; Bootsma et al. (1995) Lancet 345:1595-1599.

Synthetic double-stranded oligonucleotides (dsON) have been shown to cross-react with anti-dsDNA antibodies (U.S. Pat. No. 5,276,013). The use of dsON conjugated with non-immunogenic carriers, also referred to as platforms, has been proposed for a therapeutic approach for the treatment of SLE. For example, a tetrakis conjugate, LJP 249, composed of four dsON attached to a poly(ethylene glycol) valency platform was used to demonstrate tolerance in an immunized mouse model system (Jones et al. (1994) Bioconjugate Chem. 5:390-399).

Although overall patient prognosis in SLE has improved, treatment regimens are not ideal and lupus nephritis continues to be associated with relatively poor overall survival as compared to individuals without renal involvement in lupus (Seleznick et al. (1991) Semin. Arthritis Rheum. 21:73-80). Lupus nephritis is a primary cause of morbidity and mortality in SLE. Pistiner et al. (1991) Semin. Arthritis Rehum. 21:55-64. Management of patients with lupus nephritis often requires immunosuppression in the form of high dose systemic corticosteroids, azathioprine and/or cyclophosphamide. However, the utility of these agents can be limited by significant drug-induced toxicity, and these drugs lack specificity.

LJP 394, a tetravalent conjugate composed of four dsON attached to a platform, was shown to delay progression of renal disease and extend survival in the BXSB experimental murine lupus nephritis model (Plunkett et al. (1995) Lupus 4:S99; Coutts et al. (1996) Lupus 5:158-159). LJP 394 has also been shown to lower anti-dsDNA antibodies in human patients with SLE (Weisman et al. (1997) J. Rheumatol. 24:314-318). International Patent Application No. WO 01/41813 discloses methods of identifying lupus patients, including those with lupus nephritis, with high affinity anti-dsDNA antibodies and treatment of such patients with LJP 394. Other references discuss LJP394 in the context of a potential therapeutic agent for lupus. See Strand (2001) Lupus 10:216-221; Wallace (2001) Expert Opinion of Investigational Drugs 10:111-117; Furie et al. (2001) J. Rheumatol. 28:257-265.

Other literature describes methods which may be used in the treatment of SLE, including methods of reducing levels of circulating antibodies by inducing B cell tolerance, including, but not limited to, U.S. Pat. Nos. 5,276,013; 5,391,785; 5,786,512; 5,726,329; 5,552,391; 5,268,454; 5,606,047; 5,633,395; 5,162,515; U.S. Ser. No. 08/118,055 (U.S. Pat. No. 6,060,056); U.S. Ser. Nos. 60/088,656 and 60/103,088 (U.S. Ser. No. 09/328,199 and PCT App. No. PCT/US99/13194). See also U.S. Pat. No. 6,022,544.

All references cited herein, including patents, patent applications and publications, are hereby incorporated by reference in their entirety.

### DISCLOSURE OF THE INVENTION

The invention provides methods for treatment of systemic lupus erythematosis, particularly symptoms related to renal dysfunction (e.g., lupus nephritis or LN) in individuals with significant renal impairment, i.e., significantly impaired renal function. Accordingly, in one aspect, the invention provides methods of treating LN in an individual with SLE, said method comprising administering to the individual a conjugate comprising: (a) a non-immunogenic valency platform molecule and (b) two or more double stranded DNA epitopes, preferably polynucleotides, wherein said individual has significantly impaired renal function. In another aspect, the invention provides methods of reducing incidence of renal flares in an individual with SLE, said method comprising administering to the individual a conjugate comprising: (a) a non-immunogenic valency platform molecule and (b) two or more double stranded DNA epitopes, preferably polynucleotides, wherein said individual has significantly impaired renal function. In another aspect, the invention provides methods of treating LN in an individual, comprising selecting a SLE patient having significantly impaired renal function and administering to the individual a conjugate comprising (a) a non-immunogenic valency platform molecule and (b) two or more double stranded DNA epitopes, preferably polynucleotides. Preferably, at least one of said epitopes is bound at a high initial affinity by antibodies from the patient (that is, as described herein, at least one of the epitopes binds with high affinity to anti-double stranded DNA antibody in the individual). Preferably, all epitopes on the conjugate bind with high affinity to anti-double stranded DNA antibody in the individual. Individuals are selected for treatment in accordance with the instant methods on the basis of a diagnosis of systemic lupus erythematosis (SLE) and at least one clinical indication of significantly impaired renal function. In certain preferred embodiments, the conjugate comprises (a) a non-immunogenic valency platform molecule and (b) two or more polynucleotides comprising, consisting essentially of or consisting of the double stranded DNA sequence 5′-GTGTGTGTGTGTGTGTGTGT-3′(SEQ ID NO:1). In other embodiments, the invention provides methods of treating SLE in an individual comprising administering a conjugate described herein, wherein assessment of renal function in the individual, particularly identifying individuals based on significantly impaired renal function, is a basis for selecting individuals to receive such treatment.

In another aspect, the invention provides methods of identifying an individual who may be suitable for treatment for SLE, said treatment comprising administration of a conjugate described herein, said method comprising measuring the renal function of said individual, wherein an individual is identified by having at least one clinical indication of significantly impaired renal function. In some embodiments, the conjugate comprises (a) a non-immunogenic valency platform molecule and (b) two or more polynucleotides which specifically bind to an antibody from the individual which specifically binds to double stranded DNA, said polynucleotide comprising, consisting essentially of, or consisting of the dsDNA sequence 5′-GTGTGTGTGTGTGTGTGTGT-3′(SEQ ID NO:1).

Preferably, an individual is also selected (and in some embodiments, treated) based on affinity of anti-double stranded DNA antibodies for an epitope(s) of the conjugate. In accordance with the invention, antibody affinity is measured as the apparent equilibrium dissociation constant (K_{D}′) (or its functional equivalent) for a dsDNA epitope. In certain embodiments the individual is selected for treatment in accordance with the instant invention if the K_{D}′ (or its functional equivalent) is less than about 1.0 mg IgG per mL. Other, lower K_{D}′ values are described herein which could apply to any of the dsDNA epitopes contemplated for use in treatment, as are percentile ranking with respect to a given patient population as described herein.

The invention also provides kits for use in the methods of the invention. Such kits comprise a conjugate comprising (a) a non-immunogenic valency platform molecule and (b) two or more polynucleotides and instructions for use of the conjugate comprising a description of selecting an SLE patient having significantly impaired renal function, and administering said conjugate to the patient. The instructions may further relate to measurement of affinity of anti-dsDNA antibodies from the patient.

### BRIEF DESCRIPTION OF THE DRAWINGS

FIGS. 1A-C are graphs depicting competitive inhibition by LJP 394 of antibodies from groups of SLE patients' sera binding to ¹²⁵I-labeled dsDNA.

### MODES FOR CARRYING OUT THE INVENTION

We have discovered that administration of a conjugate comprising a non-immunogenic platform molecule and four double stranded DNA epitopes, namely, LJP 394 (having four double stranded DNA molecules with the sequence 5′-GTGTGTGTGTGTGTGTGTGT-3′(SEQ ID NO:1)) to systemic lupus erythematosis (SLE) patients with renal disease, but especially to those patients having significant impaired renal function effected significant improvement in terms of fewer renal flares as well as longer time to flare. This result of apparent superior benefit of LJP 394 treatment in patients with significantly impaired renal function was surprising and unexpected. The greatest benefit was observed in those patients having significantly impaired renal function and also having high affinity antibodies to the dsDNA epitope(s) of LJP 394. The instant invention is based upon analysis of data from a clinical trial of LJP 394 referred to as the 90-05 study, some accounts of which have been published as Linnik et al. (2000) Arth. Rheumat. 43(9 supplement):S241 (abstracts 1045 and 1046) and Alarcon-Segovia et al. (2000) Arth. Rheumat. 43(9 supplement):S272 (abstract 1231) and are described herein. Patients with significantly impaired renal function in the placebo group appeared more prone to renal flare as 60% of these patients had a renal flare, versus 20% of the intent to treat (ITT) placebo population (Example 1). LJP394 treatment appeared to reduce renal flares, especially in the high affinity patients where there was a complete absence of renal flares. These results are significant, given the intractability of treatment of significant renal impairment. With the benefit of applicants' discovery, such patients are included in treatment, or at least selected as suitable as receiving such treatment based on the condition of having significant renal impairment. In some embodiments, suitable individuals are also selected based on having high affinity antibodies with respect to an epitope(s) of the conjugate.

Accordingly, the invention provides methods of alleviating one or more symptoms of lupus nephritis (in some embodiments, reducing incidence of renal flares) in an individual, comprising administering to said individual a conjugate comprising a non-immunogenic valency platform molecule and two or more polynucleotides, at least one of said polynucleotides comprising the dsDNA epitope, wherein the individual has significantly impaired renal function. In some embodiments, the methods comprising selecting an individual suffering from SLE who has significantly impaired renal function and administering the conjugate(s) as described. Preferably, at least one of said epitopes in the conjugate is bound at a high initial affinity by antibodies from the patient. Preferably, all epitopes on the conjugate bind with high affinity to anti-double stranded DNA antibody in the individual. In accordance with the instant invention, SLE patients are selected and/or treated on the basis of the presence of significantly impaired renal function, and preferably the presence of high affinity antibodies with respect to the double stranded DNA epitope(s) of the conjugate to be used. The invention also provides methods of selecting an individual suitable for the conjugate-based treatments described herein based on assessment of renal function and selecting a patient suitable for receiving the treatment based on identifying those patients having significant renal impairment.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991) and Short Protocols in Molecular Biology (Wiley and Sons, 1999). Other useful references include Harrison's Principles of Internal Medicine (McGraw Hill; J. Isseleacher et al., eds.) and Dubois' Lupus Erythematosus (5th ed.; D. J. Wallace and B. H. Hahn, eds.; Willaims & Wilkins, 1997).

### Definitions

An individual having “significantly impaired renal function” or “significant renal impairment” is an individual exhibiting one or more clinical signs of significant renal dysfunction, as described herein. Clinical signs of renal dysfunction include anuria, oliguria, elevated blood urea nitrogen (BUN), elevated serum creatinine, clinically significant proteinuria, hematuria, reduced creatinine clearance, and other clinical indications of renal dysfunction known in the art. As described herein, generally, an individual displays significant renal impairment if any one of more of these clinical indicia are at least above the upper limit of “normal” range, as defined in the clinical arts. In some embodiments, significant renal impairment is indicated if the value exceeds the upper limit of normal by about any of the following percentages: 10, 20, 25, 30, 50, 60, 75, 100, 125, 150, 200, 250, 275, 300, 350, 400, 450, 500. As is known in the art, with respect to at least one indicia of kidney function, such as serum creatinine, an individual can have at least about 2, 3, 5, or 10 fold or greater values compared with the upper limit of normal. Generally, an individual is determined to have, or in fact has, significant renal impairment at the onset (before the individual receives the first administration), or shortly after the onset (within about 4 weeks, preferably within about 2 weeks, preferably within about 1 week, preferably within about 5 days, preferably within about 2 days, preferably within about 1 day) upon receiving the first administration), of the therapeutic methods described herein.

When significantly impaired renal function “is used as a basis” for administration of the treatment methods described herein, or selection for the treatment methods described herein, renal function is measured before and/or during treatment, and the values obtained are used by a clinician in assessing probable or likely suitability of an individual to receive treatment(s). As would be well understood by one in the art, measurement of renal function in a clinical setting is a clear indication that this parameter was used as a basis for initiating, continuing, adjusting and/or ceasing administration of the treatments described herein.

“Affinity” of an antibody from an individual for an epitope to be used, or used, in treatment(s) described herein is a term well understood in the art and means the extent, or strength, of binding of antibody to epitope. Affinity may be measured and/or expressed in a number of ways known in the art, including, but not limited to, equilibrium dissociation constant (K_{D} or K_{d}), apparent equilibrium dissociation constant (K_{D}′ or K_{d}′), and IC₅₀ (amount needed to effect 50% inhibition in a competition assay; used interchangeably herein with “I₅₀”). It is understood that, for purposes of this invention, an affinity is an average affinity for a given population of antibodies which bind to an epitope. Values of K_{D}′ reported herein in terms of mg IgG per mL or mg/mL indicate mg Ig per mL of serum, although plasma can be used.

When antibody affinity “is used as a basis” for administration of the treatment methods described herein, or selection for the treatment methods described herein, antibody affinity is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. As would be well understood by one in the art, measurement of antibody affinity in a clinical setting is a clear indication that this parameter was used as a basis for initiating, continuing, adjusting and/or ceasing administration of the treatments described herein.

An antibody affinity measured “before or upon initiation of treatment” or an “initial affinity” is antibody affinity measured in an individual before the individual receives the first administration of a treatment modality described herein and/or within at least about 4 weeks, preferably within at least about 2 weeks, preferably within at least about 1 week, preferably within at least about 5 days, preferably within at least about 3 days, preferably within at least about 2 days, preferably within at least about 1 day upon receiving the first administration of a treatment modality described herein.

A “population” is a group of individuals with lupus. For a given population (which may vary in terms of number of members, depending on the context) antibody affinities vary over a range (i.e., maximum and minimum affinities).

As used herein, “treatment” is an approach for obtaining beneficial or desired results including and preferably clinical results. For purposes of this invention, beneficial or desired clinical results include, but are not limited to, one or more of the following: alleviation of one or more symptoms, diminishment of extent of disease, stabilized (i.e., not worsening) state of disease, preventing spread of disease, preventing occurrence or recurrence of disease, decreasing, delaying or preventing the occurrence of renal “flares,” amelioration of the disease state, remission (whether partial or total), reduction of incidence of disease and/or symptoms, stabilizing (i.e., not worsening) of renal function or improvement of renal function. During lupus nephritis, which is a chronic inflammatory kidney disease, “flares” may occur. “Flares” refer to an increase in activity, generally inflammatory activity. If the activity is in the kidneys, then the flare is referred to as a “renal flare”. “Renal flares” can be identified by evaluating factors including, but not limited to, proteinuria levels, hematuria levels, and serum creatinine levels. The “treatment” of lupus nephritis may be administered when no symptoms of lupus nephritis are present, and such treatment (as the definition of “treatment” indicates) reduces the incidence of flares. Also encompassed by “treatment” is a reduction of pathological consequences of any aspect of lupus nephritis.

“SLE flares” are used herein to refer to flares (i.e. acute clinical events) which occur in patients with SLE. The SLE flares may be in various major organs, including but not limited to, kidney, brain, lung, heart, liver, and skin. SLE flares include renal flares.

“Reducing incidence” of renal flares in an individual with SLE means any of reducing severity (which can include reducing need for and/or amount of (e.g., exposure to) other drugs generally used for this conditions, including, for example, high dose corticosteroid and/or cyclophosphamide), duration, and/or frequency (including, for example, delaying or increasing time to renal flare as compared to not receiving treatment) of renal flare(s) in an individual. As is understood by those skilled in the art, individuals may vary in terms of their response to treatment, and, as such, for example, a “method of reducing incidence of renal flares in an individual” reflects administering the conjugate(s) described herein based on a reasonable expectation that such administration may likely cause such a reduction in incidence in that particular individual.

“High dose corticosteroid and/or cyclophosphamide” or “HDCC” as used herein refers to intervention with an increased dosage of corticosteroid alone or with cyclophosphamide. High dose generally refers to corticosteroids. Such intervention generally occurs upon a flare, or acute episode. Generally, for example, the increased dosage is at least a 15 mg/day and can be greater than 20 mg/day. HDCC may be administered using standard clinical protocols. A clinician may monitor a patient and determine when HDCC treatment is needed by evaluating factors including, but not limited to, proteinuria levels, hematuria levels, and serum creatinine levels. In general, patients who experience renal flares are given HDCC treatment, although this treatment is used for other aspects of lupus.

An “equivalent” or “functional equivalent” of K_{D}′ or a numerical value for K_{D}′ is a parameter or value for a parameter which also reflects affinity. For example, an equivalent of K_{D}′ is IC₅₀. As another example, an equivalent value of K_{D}′ of 0.5 could be an IC₅₀ of 200, if they reflect the same, or about the same, affinity. Determining such equivalents is well within the skill of the art and such equivalents and their determination are encompassed by this invention. Generally, reference to K_{D}′ includes reference to functional equivalents of K_{D}′.

As used herein, the singular form “a”, “an”, and “the” includes plural references unless indicated otherwise. For example, “an” antibody includes one or more antibodies.

An “epitope” is a term well-understood in the art and means any chemical moiety which exhibits specific binding to an antibody. An “epitope” can also comprise an antigen, which is a moiety or molecule that contains an epitope, and, as such, also specifically binds to antibody.

A “double-stranded DNA epitope” or “dsDNA epitope” is any chemical moiety which exhibits specific binding to an anti-double-stranded DNA antibody and as such includes molecules which comprise such epitope(s). Further discussion of double-stranded DNA epitopes suitable for the conjugates of the invention are described below. The term “epitope” also includes mimetics of double-stranded DNA itself, which are described below.

An epitope that “specifically binds” to an antibody is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. For example, a antibody that specifically binds to a double stranded DNA (dsDNA) epitope is an antibody that binds the dsDNA epitope with greater affinity, avidity, more readily, and/or with greater duration than it binds to non-polynucleotide epitopes. It is also understood by reading this definition that, for example, an antibody (or moiety or epitope) that specifically binds to a first target may or may not specifically bind to a second target. As such, “specific binding” or “specifically binding” does not necessarily require (although it can include) exclusive binding.

An “anti-double-stranded DNA antibody” or “anti-dsDNA antibody” or “double-stranded DNA antibody” or “antibodies to dsDNA”, used interchangeably herein, is any antibody which specifically binds to double-stranded DNA (dsDNA). An “anti-ds DNA antibody” can also specifically bind to a single-stranded DNA, and as such, this term includes antibodies which cross-react with single-stranded DNA, although such cross-reactivity is not required. The “ds” terminology is used in accordance with the traditional nomenclature in this field. As such, based on this definition, these antibodies could also be termed “anti DNA” antibodies. Any antibody includes an antibody of any class, such as IgG, IgA, or IgM, and the antibody need not be of any particular class. As clearly indicated in the definition of “antibody” provided herein, a “anti-double-stranded DNA antibody” encompasses any fragment(s) that exhibits this requisite functional (i.e., specific binding to dsDNA) property, such as fragments that contain the variable region, such as Fab fragments. As discussed below, it is understood that specific binding to any anti-double-stranded DNA antibody (or functional fragment) is sufficient.

The term “circulating anti-double-stranded DNA antibody”, as used herein, intends an anti-double-stranded DNA antibody which is not bound to a double-stranded DNA epitope on and/or in a biological sample, i.e., free antibody.

An “antibody” (interchangeably used in plural form) is an immunoglobulin molecule capable of specific binding to a target, such as a carbohydrate, polynucleotide or polypeptide, through at least one antigen recognition site, located in the variable region of the immunoglobulin molecule. As used herein, the term encompasses not only intact antibodies, but also fragments thereof (such as Fab, Fab′, F(ab′)₂, Fv), single chain (ScFv), mutants thereof, fusion proteins comprising an antibody portion, humanized antibodies, and any other modified configuration of the immunoglobulin molecule that comprises an antigen recognition site of the required specificity.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. For purposes of this invention, unless otherwise indicated, sequences presented herein denote double stranded sequences. For example, the polynucleotide comprising, consisting essentially of, or consisting of the double stranded sequence 5′-GTGTGTGTGTGTGTGTGTGT-3′(SEQ ID NO:1) includes the complementary polynucleotide sequence, particularly the sequence 3′-CACACACACACACACACACA-5′(SEQ ID NO:2). It is understood that the double stranded polynucleotide sequences described herein also include the modifications described herein. The backbone of the polynucleotide can comprise sugars and phosphate groups (as may typically be found in RNA or DNA), or modified or substituted sugar or phosphate groups. Alternatively, the backbone of the polynucleotide can comprise a polymer of synthetic subunits such as phosphoramidates and thus can be a oligodeoxynucleoside phosphoramidate (P—NH2) or a mixed phosphoramidate-phosphodiester oligomer. A phosphorothioate linkage can be used in place of a phosphodiester linkage. In addition, a double-stranded polynucleotide can be obtained from the single stranded polynucleotide product of chemical synthesis either by synthesizing the complementary strand and annealing the strands under appropriate conditions, or by synthesizing the complementary strand de novo using a DNA polymerase with an appropriate primer.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. For purposes of this invention, a polynucleotide is generally an isolated polynucleotide of less than about 1 kb, preferably less than about 500 base pairs (bp), preferably less than about 250 bp, preferably less than about 100 bp, preferably less than about 50 bp. However, it is understood that a polynucleotide of any size or configuration could be used as long as it exhibits the requisite binding to anti dsDNA antibody from an individual. It is further understood that a different polynucleotide (for example, in terms of size and/or sequence) other than the one that is to be, was, or will be used in treatment, as long as both polynucleotides exhibit equivalent (or convertible) binding affinities to anti-dsDNA antibodies from an individual. In other words, non-identical polynucleotides may be employed with respect to affinity determination and treatment.

Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

“Naturally occurring” refers to an endogenous chemical moiety, such as a carbohydrate, polynucleotide or polypeptide sequence, i.e., one found in nature. Processing of naturally occurring moieties can occur in one or more steps, and these terms encompass all stages of processing. Conversely, a “non-naturally occurring” moiety refers to all other moieties, i.e., ones which do not occur in nature, such as recombinant polynucleotide sequences and non-naturally occurring carbohydrates.

As used herein, the term “immunogen” means a chemical entity that elicits a humoral immune response when injected into an animal. Immunogens have both B cell epitopes and T cell epitopes.

As used herein, the term “analog” (also termed an “mimetic”) of an immunogen means a biological or chemical compound which specifically binds to an antibody to which the immunogen specifically binds. As such a “double-stranded DNA epitope” includes mimetics of naturally-occurring double-stranded DNA. An “analog” or “mimetic” shares an epitope, or binding specificity, with double-stranded DNA. An analog may be any chemical substance which exhibits the requisite binding properties, and thus may be, for example, a simple or complex organic or inorganic molecule; a polypeptide; a polynucleotide; a carbohydrate; a lipid; a lipopolysaccharide; a lipoprotein, or any combination of the above, including, but not limited to, a polynucleotide-containing polypeptide; a glycosylated polypeptide; and a glycolipid. The term “analog” encompasses the term “mimotope”, which is a term well known in the art.

An “individual” is a vertebrate, preferably a mammal, more preferably a human. Mammals include, but are not limited to, farm animals, sport animals, pets, primates, mice and rats.

“Inducing tolerance” or “inducing immunotolerance” means a reduction and/or stabilization of the extent of an immune response to an immunogen, and, as such, means immune unresponsiveness (or at least a reduction in the extent of an immune response) at the organismal level and unresponsiveness (e.g., anergy) and/or apoptosis at the cellular level. An “immune response” may be humoral and/or cellular, and may be measured using standard assays known in the art. For purposes of this invention, the immune response is generally reflected by the presence of, and/or the levels of, anti-double-stranded DNA antibodies. Quantitatively the reduction (as measured by reduction in antibody production and/or levels) is at least about 15%, preferably at least about 25%, more preferably at least about 50%, more preferably at least about 75%, more preferably at least about 90%, even more preferably at least about 95%, and most preferably 100%. It is understood that the tolerance is antigen-specific, and applies for purposes of the invention to those individuals having anti-double-stranded DNA antibodies. “Inducing tolerance” also includes slowing and/or delaying the rate of increase of antibody level.

As used herein, the term “B cell anergy” intends unresponsiveness of those B cells requiring T cell help to produce and secrete antibody and includes, without limitation, clonal deletion of immature and/or mature B cells and/or the inability of B cells to produce antibody. “Unresponsiveness” means a therapeutically effective reduction in the humoral response to an immunogen. Quantitatively the reduction (as measured by reduction in antibody production) is at least 50%, preferably at least 75% and most preferably 100%.

An “effective amount” (when used in the lupus context, or in the antibody-mediated pathology context) is an amount sufficient to effect beneficial or desired results including clinical results. An effective amount can be administered in one or more administrations. For purposes of this invention, an effective amount of conjugate described herein (or a composition comprising a conjugate) an amount sufficient to reduce circulating levels of anti-double-stranded DNA antibodies, preferably by inducing tolerance, particularly with respect to anti-double-stranded DNA antibodies. In terms of treatment, an “effective amount” of conjugate described herein (or a composition comprising a conjugate) is an amount sufficient to palliate, ameliorate, stabilize, reverse, slow or delay progression of or prevent systemic lupus erythematosis (SLE), including the progressive inflammatory degeneration of the kidneys that results from SLE (i.e., lupus nephritis).

A “stable complex” formed between any two or more components in a biochemical reaction, refers to a duplex or complex that is sufficiently long-lasting to persist between formation of the duplex or complex and subsequent detection, including any optional washing steps or other manipulation that may take place in the interim.

An “isolated” or “purified” polypeptide or polynucleotide is one that is substantially free of the materials with which it is associated in nature. By substantially free is meant at least 50%, preferably at least 70%, more preferably at least 80%, even more preferably at least 90% free of the materials with which it is associated in nature.

As used herein “valency platform molecule” means a nonimmunogenic molecule containing sites which allow the attachment of a discrete number of epitopes and/or mimetic(s) of epitopes. A “valency” of a conjugate or valency platform molecule indicates the number of attachment sites per molecule for a double-stranded DNA epitope(s). Alternatively, the valency of a conjugate is the ratio (whether absolute or average) of double-stranded DNA epitope to valency platform molecule.

“Nonimmunogenic”, when used to describe the valency platform molecule, means that the valency platform molecule fails to elicit an immune response (i.e., T cell and/or B cell response), and/or fails to elicit a sufficient immune response, when it is administered by itself to an individual. The degree of acceptable immune response depends on the context in which the valency platform molecule is used, and may be empirically determined.

An epitope which is “conjugated” to a valency platform molecule is one that is attached to the valency platform molecule by covalent and/or non-covalent interactions.

An “epitope-presenting valency platform molecule” is a valency platform molecule which contains attached, or bound, epitopes, at least some of which (at least two of which) are able to bind an antibody of interest.

A “biological sample” encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides. The term “biological sample” encompasses a clinical sample, and also includes cells in culture, cell supernatants, cell lysates, serum, plasma, biological fluid, and tissue samples.

“In conjunction with” refers to administration of one treatment modality in addition to another treatment modality, such as administration of a conjugate described herein in addition to administration of corticosteroid cyclophosphamide immunosuppressants (or other immunosuppressant therapy) to the same individual. As such, “in conjunction with” refers to administration of one treatment modality before, during or after delivery of the other treatment modality to the individual.

“Receiving treatment” includes initial treatment and/or continuing treatment.

“Comprising” means including.

### Methods of Treatment

The invention provides methods for treatment of systemic lupus erythematosis (SLE), particularly symptoms related to renal dysfunction (e.g., lupus nephritis). Accordingly, in one aspect, the invention provides methods of treating lupus nephritis (LN) in an individual, comprising administering to the individual an epitope presenting conjugate comprising (a) a non-immunogenic valency platform molecule and (b) two or more double stranded DNA epitopes, preferably polynucleotides, wherein the individual has significantly impaired renal function (as indicated by measuring one or more clinical indicia of renal function as known in the art and/or described herein). Preferably, at least one of said epitopes is bound at a high initial affinity by at least one anti dsDNA antibody from the patient (that is, as described herein, at least one population or type of antibodies from the individual binds at high affinity to an epitope(s) of the conjugate). In some embodiments, the methods comprising selecting an SLE patient having significantly impaired renal function. Individuals having SLE, or who are suspected of having SLE, are selected for treatment in accordance with the instant methods on the basis of the presence of at least one clinical indication of significantly impaired renal function. Preferably, selection is also based upon the presence of antibodies which bind to a double-stranded DNA (dsDNA) epitope at high affinity in the individual. Accordingly, in some embodiments of the invention, the methods include an additional step of assessing the affinity of the individual's antibodies for a dsDNA epitope present in the conjugate before or upon initiation of treatment, as described in, for example, PCT/US00/42307 (WO01/41813).

In certain embodiments, the methods of the invention include an reassessment step, in which the affinity of the individual's antibodies for at least one of the dsDNA epitope(s) on the conjugate is remeasured. This remeasurement may serve as the basis for continuing, or discontinuing, the treatment. In such embodiments including a reassessment step, treatment is generally, but not necessarily, continued if the affinity of the individual's antibodies has decreased, or generally, but not necessarily, discontinued if the affinity of the individual's antibodies has failed to decrease.

In some embodiments, a conjugate is administered in an amount sufficient to reduce incidence of, or likelihood of, renal flares particularly in individuals having significantly impaired renal function. Accordingly, the invention provides methods of treating LN in an individual, comprising administering to the individual an epitope presenting conjugate comprising (a) a non-immunogenic valency platform molecule and (b) two or more double stranded DNA epitopes, preferably polynucleotides, wherein the individual has significantly impaired renal function. In some embodiments, the methods comprise selecting an individual with LN having significantly impaired renal function. Preferably, at least one of said epitopes is bound at a high initial affinity by at least one antibody from the patient. In some embodiments, the apparent equilibrium dissociation constant (K_{D}′) for the polynucleotide in the conjugate with respect to the antibody from the individual before or upon initiation of treatment is less than about 1.0 mg IgG per mL. Preferably, the K_{D}′ value is used as a basis for selecting the individual to receive the treatment. In other embodiments, the K_{D}′ is less than about any of the following: 0.8; 0.7; 0.6; 0.5; 0.4; 0.3; 0.2; 0.1; 0.09; 0.08; 0.07; 0.06 0.05; 0.025.

In some embodiments, a conjugate as described herein is administered in an amount sufficient to reduce the exposure (i.e., dose and/or length of treatment) of an individual to corticosteroid and/or cyclophosphamide immunosuppressive therapy that would otherwise be administered in the absence of administering the conjugate. This is significant, as this type of immunotherapy is toxic. Accordingly, the invention provides methods of treating lupus nephritis comprising administering to the individual a conjugate comprising (a) a non-immunogenic valency platform molecule and (b) two or more double stranded DNA epitopes, preferably polynucleotides, wherein the individual has significantly impaired renal function. In some embodiments, the methods comprising selecting an individual with LN having significantly impaired renal function. Preferably, at least one of said epitopes is bound at a high initial affinity by at least one antibody from the patient.

The invention also provides methods of treating SLE, preferably lupus nephritis, comprising administering a conjugate described herein in conjunction with corticosteroid and/or cyclophosphamide, wherein the individual has (or is suspected of having) SLE, and wherein the individual has significantly impaired renal function. In some embodiments, the methods comprise selecting an individual having significantly impaired renal function. The conjugate is generally administered in an amount effective to reduce antibody affinity for the epitope in the conjugate, although any amount that effects a desired result (such as reduction of incidence of renal flares, or any other description in the definition of “treatment”) in conjunction with corticosteroid and/or cyclophosphamide is acceptable. Preferably, the conjugate is LJP 394, which is described herein. Methods of administering corticosteroid and/or cyclophosphamide are known in the art. Reducing the dosage of corticosteroid and/or cyclophosphamide therapy (which reduces the dependence on administration of these drugs and in effect delays administration of these drugs) can be assessed by, for example, comparing to known and/or established averages of dosage (in terms of amount and/or intervals) generally given over time which are known in the art.

In certain embodiments, the affinity of the antibodies from the patient is quantified as the apparent equilibrium dissociation constant (K_{D}′) for the epitope(s) in the conjugate. In such embodiments, the individual's antibodies are considered to have a high affinity if the K_{D}′ with respect to the dsDNA epitope(s) before or upon initiation of treatment is less than about 1.0 mg IgG per mL. In other embodiments, the K_{D}′ is less than about any of the following: 0.8; 0.7; 0.6; 0.5; 0.4; 0.3; 0.2; 0.1; 0.09; 0.08; 0.07; 0.06; 0.05; 0.025. It should be noted that measurement of affinity, either represented by measuring K_{D}′ or by some other method, either before or during treatment is strong, if not conclusive, indication that this parameter was a basis for selecting the individual to receive treatment.

### Selection of Individuals for Treatment

The instant method involves treating and/or selecting an individual who has, or is suspected of having, systemic lupus erythematosus (SLE) who also has significantly impaired renal function. The symptoms of SLE are well known in the art, and it is well within the knowledge of those of ordinary skill in the art to identify individuals having, or who are suspected of having, SLE. Within the group of individuals having, or being suspected of having, SLE, selecting those having significantly impaired renal function may be on the basis of any clinical indication of significant renal impairment known in the art, including, but not limited to, anuria, oliguria, elevated serum creatinine levels, elevated BUN, proteinuria, hematuria (occult or gross), reduced creatinine clearance, impaired glomeral filtration, and the like. As will be apparent to one of skill in the art, a diagnosis of renal dysfunction, such as a diagnosis of subacute glomerulonephritis, nephrotic syndrome, or mild to severe nephritis, will also identify a significant impairment of renal function and thus serve as a basis for treating that individual and/or selection of the individual for treatment in accordance with the instant methods.

As will be apparent, the quantitative level of a particular clinical parameter that indicates a significant impairment of renal function will depend on the particular clinical parameter. Proteinuria is easily detected at a ‘screening’ level using colorimetric “dipstick” testing of urine, and can be followed up by more sensitive and accurate laboratory testing. Preferably, when the presence of a significant impairment of renal function is identified by proteinuria, an individual is considered to have significantly impaired renal function when at least about 500 mg of protein is excreted in the urine per day, more preferably at least about (i.e., greater than or equal to about) 1.5, 2, 2.5, 3, 3.5, 5.0, 6.0, 7.0, 8.0, 9.0, or 10 grams of protein per day. When serum creatinine is used as the indicator of significant impairment of renal function, an individual will be considered to have significantly impaired renal function when serum creatinine levels are at least about (i.e., greater than or equal to about) 1.5, 2, 2.5 3.0, 3.5, 4.0, 5.0, 6.0, 7.0, 8.0, 9.0, or 10 milligrams per deciliter (mg/dL).

As will be understood by one of skill in the art, administration of a conjugate comprising a non-immunogenic valency platform molecule and two or more double stranded DNA epitopes, preferably polynucleotides, and preferably wherein at least one of said epitopes is bound at a high initial affinity by antibodies from the patient entails assessing antibody affinity from an individual in those embodiments in which selection is based on antibody affinity, wherein said individual has, or is suspected of having, SLE. For purposes of this invention; (a) the affinity in question is with respect to an individual's antibodies, that is, antibodies obtained from that individual; (b) the antibody for which affinity is measured is an antibody associated with, and/or implicated in SLE; and (c) the binding of interest is binding of antibody to an epitope which binds to the antibody(ies), generally the epitope to be used in the proposed treatment, as described herein (i.e., a dsDNA epitope), or binding which correlates with binding of the epitope(s) to be used in the proposed treatment.

For all embodiments of the invention which use or are directed to K_{D}′, whether screening, treatment, monitoring, or any other methods directed to assessing affinity, it is understood that other, equivalent values can be measured and used, and are encompassed by this invention. For example, as discussed below, there are a number of methods known in the art which can measure (and express) affinity of antibodies from an individual for an epitope to be used for treatment (in the context of this invention, a double stranded DNA epitope). As is understood and conveyed by this disclosure, affinity may be measured using any epitope whose binding to the dsDNA antibody correlates with binding of the epitope(s) to be used in the proposed treatment (for example, a single-stranded counterpart of a double-stranded polynucleotide). K_{D}′ is one of these parameters, and equivalent parameters can be measured and used in this invention. Further, with respect to K_{D}′ cut-off values reported herein, the basis of this finding was administering about 100 mg of LJP 394 conjugate about once a week.

Measurement of affinity, either represented by measuring K_{D}′ or by some other method, either before or during treatment is strong, if not conclusive, indication that this parameter was a basis for selecting the individual to receive (and/or continue to receive) treatment. Accordingly, with respect to all treatment methods described herein, and as the definition for “is used as a basis” states, other embodiments include (1) assessing, or measuring, the affinity as described herein (and preferably selecting an individual suitable for receiving (including continuing to receive) treatment); and (2) administering the treatment(s) as described herein. As described herein, in some embodiments, more than one measurement is made, when change (if any) in affinity is assessed.

Antibody affinity may be measured using methods known in the art which assess degree of binding of a DNA epitope to an antibody. Generally, these methods comprise competition assays and non-competition assays. With respect to polynucleotide epitopes (which will be used in a conjugate to be administered), affinity may be measured using polynucleotide alone or polynucleotide-containing conjugates (as long as the polynucleotide and conjugate give equivalent, or at least convertible, values). Affinity may be measured using the epitope (or a molecule or moiety comprising the epitope) used in the conjugate; alternatively, a similar, non-identical epitope may be used, as long as its affinity may be at least correlated to the affinity of the epitope used in the conjugate, so that a meaningful measurement of affinity may be obtained.

In a competition assay, varying concentrations of antibody or epitope are reacted with epitope or antibody, and results may be expressed in terms of amount of antibody (generally in terms of concentration) required to reach half-maximal binding, generally designated as IC₅₀.

Another convenient way to express affinity is apparent equilibrium dissociation constant, or K_{D}′, which reflects the titer-weighted average affinity of the antibody for the antibody-binding epitope on the conjugate. Antibody is generally obtained from whole blood and measured, by plasma, serum, or as an IgG fraction, and the affinity of this fraction for the conjugate is measured. Methods of obtaining IgG fractions are known in the art and are described herein. One preferred way to measure affinity is to measure K_{D}′ based on a surface plasmon resonance assay as described in Example 2.

Another way to measure affinity is by kinetic (i.e., non-equilibrium) analysis, methods of which are known in the art. Preferably, rate of dissociation (i.e., off rate) of antibody from epitope is measured.

In preferred embodiments, the affinity of the individual's antibodies for the dsDNA epitope(s) (whether measured directly using the epitope itself or using a moiety/epitope the affinity of which may be correlated to the affinity of the epitope used in the conjugate) is measured as the apparent equilibrium dissociation constant (K_{D}′) for the dsDNA epitope(s) in the conjugate before or upon initiation of treatment is less than about (in some embodiments, less than or equal to about) 1.0 mg IgG per mL. In other embodiments, the K_{D}′ is less than about (in some embodiments, less than or equal to about) any of the following: 0.8; 0.7; 0.6; 0.5; 0.4; 0.3; 0.2; 0.1; 0.09; 0.08; 0.07; 0.06; 0.05; 0.025. In some embodiments, K_{D}′ is less than about (in some embodiments, less than or equal to about) 0.8 mg IgG per mL. In some embodiments, K_{D}′ is less than or equal to about (in some embodiments, less than or equal to about) 0.5 mg IgG per mL. In some embodiments, K_{D}′ is less than about (in some embodiments, less than or equal to about) 0.1 mg IgG per mL.

In some embodiments, an individual is considered to have high affinity for a dsDNA epitope if the antibody affinity of the individual is in a relatively high percentile ranking of affinity compared to a population. For example, there is a range of antibody affinities over a given patient population, and individuals considered to have high affinity for a dsDNA epitope can be identified based on a percentile ranking of antibody affinity with respect to this population. Accordingly, in some embodiments, an individual is considered to have high affinity antibodies if the antibody affinity relative to the dsDNA epitope(s) for that individual is greater than about the 20th percentile (i.e., in about the top 80% of affinities for that population), and considered to not have high affinity antibodies (i.e., is not selected for treatment in accordance with the invention) if the individual's antibody affinity is in or below the 20th percentile. In other embodiments, an individual is included in treatment, or identified as suitable to receive treatment, if the antibody for that individual is greater than about the 50th percentile for that population. In some embodiments, the individual is considered to have high affinity antibody if the affinity is greater than the 70th, 75th, 80th, 85th, 90th, or 95th percentile. A population may be about, or alternatively at least about any of the following, in terms of number of individuals measured: 10, 15, 20, 25, 30, 50, 60, 75, 100, 125, 150, 175, 200, 225, 250, 300, 400, 500. Preferably, a sufficient number of individuals are measured to provide a statistically significant population, which can be determined by methods known in the art. An upper limit of a population may be any number, including those listed.

Affinity may or may not change over the course of treatment. In some embodiments which include a step wherein the individual's antibody affinity for the dsDNA epitope(s) is remeasured after initiation of the treatment, the treatment may be continued if the average affinity of the individual's antibodies for the dsDNA epitope(s) is decreased by at least about 15%, preferably at least about 20%, more preferably at least about 25%, more preferably at least about 40%, more preferably at least about 50%, compared to the affinity measured before or at initiation of treatment, or may be discontinued if the antibody affinity has not decreased by at least about 15% (preferably at least about 20%, more preferably at least about 25%, more preferably at least about 40%, more preferably at least about 50%). For these embodiments, antibody affinity is measured after initiation of treatment (for comparison to antibody affinity before or upon initiation of treatment) at least about 4 weeks, preferably at least about 6 weeks, more preferably at least about 10 weeks, more preferably at least about 12 weeks, after initiation of treatment. In other embodiments, treatment may be continued if antibody affinity is decreased at least about any of the following (as compared to antibody affinity before or upon initiation of treatment): 40%, 50%, 75%, 100%, 200%, 500%. Preferably, antibody affinity is measured as the K_{D}′. As is understood by those of skill in the art, K_{D}′ values are inversely proportional to the affinity of the antibodies measured. Accordingly, in some embodiments, when K_{D}′ values are used to measure antibody affinity, treatment may be continued if the K_{D}′ increases by at least about 15%, and may be continued if K_{D}′ is increased at least about any of the following (as compared to antibody affinity before or upon initiation of treatment): 40%, 50%, 75%, 100%, 200%, 500%.

When antibody affinity is assayed using surface plasmon resonance, a reduction in affinity of at least about 15%, preferably at least about 20%, more preferably at least about 25%, more preferably at least about 40%, more preferably at least about 50% indicates responsiveness and that continuation of the treatment is indicated. For a competitive Farr assay, the same reductions in affinity generally apply. For other assays, the change can be at least about any of the above percentages, and further can be at least about any of the following percentages: 75%, 100%, 150%, 200%, 250%, 300%, 350%, 400%, 450%, 500%.

The invention also provides methods of identifying an individual suitable for receiving the treatment(s) described herein based on significant impairment of renal function. These methods may be practiced independently of the treatment methods, and may be practiced by a skilled technician other than a medical doctor, using equipment and/or techniques of the art.

Accordingly, in some embodiments, the invention provides methods of identifying an individual who may be suitable for treatment for SLE, especially lupus nephritis, said treatment comprising administration of a conjugate comprising (a) a non-immunogenic valency platform molecule and (b) two or more dsDNA epitopes, preferably polynucleotides which specifically bind to an antibody from the individual which specifically binds to double stranded DNA, said method comprising assessing renal function in the individual, wherein an individual is identified by having significant renal impairment by any of the criteria described herein and/or known in the art. In some embodiments, the screening (selection) also involves measuring initial antibody affinity, as described herein. Generally, a higher affinity “cut-off” (for example, as indicated by a lower K_{D}′ value) would provide a higher degree of certainty with respect to likely success of treatment.

### Administration of Conjugates

Various formulations of epitope-presenting conjugate(s) may be used for administration, and, as such, the methods of this invention include administering a composition comprising any conjugate(s) described herein. In some embodiments, the epitope-presenting conjugate(s) may be administered “neat” (e.g., dissolved in pure water, such as USP water for injection). In some embodiments, the compositions comprise a conjugate(s) and a pharmaceutically acceptable excipient, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington's Pharmaceutical Sciences 19th Ed. Mack Publishing (1995).

Generally, these compositions are formulated for administration by injection (e.g., intraperitoneally, intravenously, subcutaneously, intramuscularly, etc.). Accordingly, these compositions are preferably combined with pharmaceutically acceptable vehicles such as saline, Ringer's solution, dextrose solution, and the like, and, as is understood in the art, are usually sterile to be suitable for injection, especially in humans. Generally, the conjugate will normally constitute about 0.01% to 10% by weight of the formulation due to practical, empirical considerations such as solubility and osmolarity. The particular dosage regimen, i.e., dose, timing and repetition, will depend on the particular individual and that individual's medical history. Generally, a dose of about 1 μg to about 100 mg conjugate/kg body weight, preferably about 100 μg to about 10 mg/kg body weight, preferably about 150 μg to about 5 mg/kg body weight, preferably about 250 μg to about 1 mg conjugate/kg body weight. Empirical considerations, such as the half life, generally will contribute to determination of the dosage. Other dosages, such as about 50 to 100 mg per week, 50 to 250 mg per week, and 50 to 500 mg per week (with any value inbetween the lower and upper limit of these ranges) are also contemplated. Example 1 provides an example of a dosing regimen. If used as a toleragen, conjugate may be administered daily, for example, in order to effect antibody clearance (pheresis), followed by less frequent administrations, such as two times per week, once a week, or even less frequently. Frequency of administration may be determined and adjusted over the course of therapy, and is based on maintaining tolerance (i.e., reduced or lack of immune response to dsDNA). Other appropriate dosing schedules may be as frequent as continuous infusion to daily or 3 doses per week, or one dose per week, or one dose every two to four weeks, or one dose on a monthly or less frequent schedule depending on the individual or the disease state. Repetitive administrations, normally timed according to B cell turnover rates, may be required to achieve and/or maintain a state of humoral anergy. Such repetitive administrations generally involve treatments of about 1 μg to about 10 mg/kg body weight or higher every 30 to 60 days, or sooner, if an increase in anti-dsDNA antibody level is detected. Alternatively, sustained continuous release formulations of the compositions may be appropriate. Various formulations and devices for achieving sustained release are known in the art. In some embodiments, LJP 394 is formulated as a sterile, colorless liquid in an isotonic phosphate-buffered saline solution for intravenous (IV) administration. Each 1 mL of solution contains 50 mg of LJP 394, 1.9 mg Na2HPO4*7H20, 0.30 mg NH₂PO₄*H₂0, and 5.8 mg NaCl in water for Injection, USP (pH 6.8-8.0). The formulation contains no preservatives. Other formulations are designed to be 20 mg/mL, 10 mg/mL, and 1 mg/mL of LJP 394. The formulations are preferably stored at cooler temperatures, such as 2 to 8° C. In other embodiments, each 1 mL of solution contains 50 mg of LJP 394, 1.9 mg Na₂HPO₄*7H₂0, 0.30 mg NH₂PO₄*H₂0, and 8.0 mg NaCl in water for Injection, USP (pH 6.8-8.0).

Other formulations include those suitable for oral administration, which may be suitable if the conjugate is able to cross the mucosa. Similarly, an aerosol formulation may be suitable.

Other formulations include suitable delivery forms known in the art including, but not limited to, carriers such as liposomes. Mahato et al. (1997) Pharm. Res. 14:853-859. Liposomal preparations include, but are not limited to, cytofectins, multilamellar vesicles and unilamellar vesicles.

In some embodiments, more than one conjugate may be present in a composition. Such compositions may contain at least one, at least two, at least three, at least four, at least five different conjugates. Such “cocktails”, as they are often denoted in the art, may be particularly useful in treating a broader range of population of individuals. They may also be useful in being more effective than using only one (or fewer than are contained in the cocktail) conjugate(s).

The compositions may be administered alone or in conjunction with other forms of agents that serve to enhance and/or complement the effectiveness of a conjugate of the invention, including, but not limited to, anti-T cell treatments. Such treatments usually employ agents that suppress T cells such as steroids or cyclosporin. Other agents are corticosteroid and/or cyclophosphamide immunosuppressive therapy.

Detection and measurement of indicators of efficacy are generally based on measurement of anti-double-stranded DNA antibody and/or clinical symptoms associated with SLE, especially lupus nephritis, which are known in the art.

Lupus nephritis (kidney glomerulonephritis or kidney inflammation) is characterized by a progressive loss of kidney function culminating in renal failure. Lupus nephritis is characterized by hematuria, decreased urine output, elevated blood urea nitrogen levels, elevated serum creatinine levels, hypertension, and proteinuria. Accordingly, these parameters can be monitored as a means of monitoring kidney degeneration. In preferred embodiments, a conjugate(s) is administered such that one or more symptoms associated with lupus nephritis is alleviated (such as reduction of incidence), as described herein.

### Treatment Modalities: Conjugates

The instant invention relates to a conjugate comprising an non-immunogenic valency platform molecule and at least two (i.e., two or more) dsDNA epitopes, preferably polynucleotides which bind to anti-dsDNA antibody from the individual. Preferably, the polynucleotide is double stranded DNA, preferably the sequence 5′-GTGTGTGTGTGTGTGTGTGT-3′(SEQ ID NO:1). In some embodiments, the polynucleotide comprises this sequence ((GT)₁₀), or consists essentially of this sequence.

dsDNA Epitope

Double-stranded DNA (dsDNA) epitopes for use in the conjugates of the present invention may be any chemical moiety which specifically binds to a dsDNA antibody. In particular, epitopes of interest include those that bind the anti-polynucleotide (particularly anti-DNA, including anti-double stranded DNA) antibodies that occur in systemic lupus erythematosis. Generally, but not necessarily, the dsDNA epitopes used are polynucleotides, preferably DNA (including DNA analogs).

Examples of suitable epitopes include, but are not limited to, those that bind to lupus anti-DNA antibodies (see U.S. Pat. Nos. 5,162,515; 5,391,785; 5,276,013; 5,786,512; 5,726,329; 5,552,391; 5,268,454; 5,633,395; 5,606,047).

The suitability of particular epitopes for binding antibodies according to this invention can be identified and/or confirmed using techniques known in the art and described herein. For example, to select the optimum epitope from a library of small drug molecules believed to mimic the dsDNA epitope for SLE, a family of platforms can be constructed in which each of the candidates is alternatively displayed on a similar platform molecule. The composition is then tested for efficacy. For example, for in vivo use, an animal model is used in which there are circulating anti-DNA antibodies, such as, for example, the BXSB mouse model system. The animals can be immunized with an appropriate epitope to initiate the antibody response, if necessary. Test candidates assembled onto a platform are then used to treat separate animals, either by administration, or by ex vivo use, according to the intended purpose. The animals are bled before and after treatment, and the antibody levels in plasma are determined by standard immunoassay as appropriate for the specific antibody. Efficacy of the candidates is then assessed according to antibody affinity assays designed to indicate antibodies specific for the epitope being tested. Appropriate affinity assays are described herein.

Polynucleotides may be screened for binding activity with antisera containing the antibodies of interest, for example, SLE antisera, by the assays described in the examples and known in the art. Examples of such assays include competitive affinity assays, for example, a competitive Farr assay and/or a competitive ELISA assay, and/or non-competitive, equilibrium affinity assay, such as the surface plasmon resonance (for example, using BIACORE®) based assay described herein.

A competitive Farr assay in which binding activity may be expressed as IC₅₀ (the polynucleotide concentration in molar nucleotides resulting in half-maximal inhibition) is an exemplary assay. Polynucleotide duplexes having an IC₅₀ of less than about 500 nM, preferably less than 50 nM, are deemed to have significant binding activity and are, therefore, useful for making the conjugates of this invention.

Another appropriate assay is the non-competitive, equilibrium affinity assay described herein, in which a titer-weighted affinity is determined.

It is understood that, for purposes of this invention, more than one type of dsDNA epitope(s) may be used in preparing a conjugate. Alternatively, one type (i.e., one chemical species) of an dsDNA epitope may be used. If a polynucleotide (such as dsDNA) is used, generally the length is greater than about 10 base pairs (bp), more preferably greater than about 15 bp, more preferably greater than or equal to about 20 bp. Generally, but not necessarily, the length is less than about 1 kb, preferably less than about 500 bp, preferably less than about 100 bp.

### Valency Platform Molecules

Any of a variety of non-immunogenic valency platform molecules (also called “platforms”) may be used in the conjugates of the invention. Many have been described in the art, such as polymers, and need not be described herein. Any non-immunogenic, acceptably low to non-toxic molecule which provides requisite attachment sites such that the conjugate may act to bind circulating anti-ds DNA antibody and/or induce B cell anergy and/or apoptosis in cells producing these antibodies may be used. Preferably, the conjugates comprise a chemically defined valency platform molecule in which a precise valency (as opposed to an average) is provided. Accordingly, a defined valency platform is a platform with defined structure, thus a defined number of attachment points and a defined valency. Certain classes of chemically defined valency platforms, methods for their preparation, conjugates comprising them and methods for the preparation of such conjugates suitable for use within the present invention include, but are not limited to, those described in the U.S. Pat. Nos. 5,162,515; 5,391,785; 5,276,013; 5,786,512; 5,726,329; 5,268,454; 5,552,391; 5,606,047; 5,663,395; and 6,060,056; and in commonly-owned U.S. Ser. No. 60/111,641 (U.S. Ser. No. 09/457,607 and PCT App. No. PCT/US99/29339); 60/138,260 (U.S. Ser. No. 09/590,592 and PCT App. No. PCT/US00/15968), U.S. Ser. No. 09/457,913 (PCT App. No. PCT/US99/29338), U.S. Ser. No. 09/457,607 (PCT/US99/29339) and U.S. Ser. No. 09/877,387 (PCT/US01/18446), all of which are hereby incorporated by reference.

A platform may be proteinaceous or non-proteinaceous (i.e., organic). Examples of proteinaceous platforms include, but are not limited to, albumin, gammaglobulin, immunoglobulin (IgG) and ovalbumin. Borel et al. (1990) Immunol. Methods 126:159-168; Dumas et al. (1995) Arch. Dematol. Res. 287:123-128; Borel et al. (1995) Int. Arch. Allergy Immunol. 107:264-267; Borel et al. (1996) Ann. N.Y. Acad. Sci. 778:80-87.

The valency of a chemically-defined valency platform molecule within the present invention can be predetermined by the number of branching groups added to the platform molecule. Suitable branching groups are typically derived from diamino acids, triamines, and amino diacids.

Preferred valency platform molecules are biologically stabilized, i.e., they exhibit an in vivo excretion half-life often of hours to days to months to confer therapeutic efficacy, and are preferably composed of a synthetic single chain of defined composition. They generally have a molecular weight in the range of about 200 to about 200,000, preferably about 200 to about 50,000 (or less, such as 30,000). Examples of valency platform molecules within the present invention are polymers (or are comprised of polymers) such as polyethylene glycol (PEG), poly-D-lysine, polyvinyl alcohol, polyvinylpyrrollidone, D-glutamic acid and D-lysine (in a ratio of 3:2). Preferred polymers are based on polyethylene glycols (PEGs) having a molecular weight of about 200 to about 8,000, or, in some embodiments, about 200 to about 10,000. In other embodiments, the molecular weight can range between about 40,000 to about 100,000; with a range of about 10,000 to about 20,000 as preferable. Other suitable platform molecules for use in the conjugates of the invention are albumin and IgG. Valency platform molecules should be of a size such that a conjugate made with the valency platform does not become a T cell independent immunogen.

Preferred valency platform molecules suitable for use within the present invention are the chemically-defined valency platform molecules disclosed, for example, in co-owned U.S. Pat. No. 5,552,391, hereby incorporated by reference. These platforms generally have low polydispersity. Particularly preferred homogeneous chemically-defined valency platform molecules suitable for use within the present invention are derivatized 2,2′-ethylenedioxydiethylamine (EDDA) and triethylene glycol (TEG). The AHAB-TEG platform used for LJP 394 (a monodisperse platform) is described below.

In some embodiments, the valency platform molecules have the advantage of having a substantially homogeneous (i.e., uniform) molecular weight (as opposed to polydisperse molecular weight). Accordingly, a population of these molecules (or conjugates thereof) are substantially monodisperse, i.e., have a narrow molecular weight distribution. A measure of the breadth of distribution of molecular weight of a sample of a platform molecule (such as a composition and/or population of platform molecules) is the polydispersity of the sample. Polydispersity is used as a measure of the molecular weight homogeneity or nonhomogeneity of a polymer sample. Polydispersity is calculated by dividing the weight average molecular weight (Mw) by the number average molecular weight (Mn). The value of Mw/Mn is unity for a perfectly monodisperse polymer. Polydispersity (Mw/Mn) is measured by methods available in the art, such as gel permeation chromatography. The polydispersity (Mw/Mn) of a sample of valency molecules is preferably less than about 2, more preferably, less than about 1.5, or less than about 1.2, less than about 1.1, less than about 1.07, less than about 1.02, or, e.g., about 1.05 to 1.5 or about 1.05 to 1.2. Typical polymers generally have a polydispersity of about 2-5, or in some cases, 20 or more. Advantages of the low polydispersity property of these valency platform molecules include improved biocompatibility and bioavailability since the molecules are substantially homogeneous in size, and variations in biological activity due to wide variations in molecular weight are minimized. The low polydispersity molecules thus are pharmaceutically optimally formulated and easy to analyze. Accordingly, in some embodiments, the valency platform molecules have very low polydispersity, and, in some embodiments are monodisperse.

Preferred platforms for dsDNA epitopes are tetrabromoacetyl compounds, and other tetravalent and octavalent valency platform molecules, such as those described in Jones et al. (1995) J. Med Chem. 38:2138-2144; and U.S. Patent references provided above.

Additional suitable valency platform molecules include, but are not limited to, tetraaminobenzene, heptaaminobetacyclodextrin, tetraaminopentaerythritol, 1,4,8,11-tetraazacyclotetradecane (Cyclam) and 1,4,7,10-tetraazacyclododecane (Cyclen).

In some embodiments, a platform having a defined number of attachment sites also comprises a (one or more) polyethylene oxide group, as described, for example, in U.S. patents and patent applications described above as well as U.S. Ser. No. 09/877,387, filed Jun. 7, 2001 (PCT/US01/18446). The molecular weight of PEG can be any molecular weight, including, but not limited to, greater than about 200, 500, 1000, 2000, 5000, 10,000, 15,000, 18,000, 22,000, 40,000, 50,000, 80,000, 100,000 Daltons. In one embodiment, in the valency platform molecule, the high molecular weight polyethylene oxide group has the formula:

—(CH₂CH₂O)ₙ—

wherein n is greater than 500; n is greater than 400; n is greater than 500; n is greater than 600; n is greater than 700; or n is greater than 800. In another embodiment, the valency platform molecule comprises a core group and at least three arms wherein each arm comprises a terminus. The core group and/or the arms may comprise a high molecular weight polyethylene oxide group. The high molecular weight polyethylene oxide group also may be attached to the core or arm. In some embodiments, a composition comprising the valency platform molecules is provided, wherein the molecules have a polydispersity less than 1.2. In another embodiment, the valency platform molecule may comprise at least three reactive conjugating groups such as hydroxyl, thiol, isocyanate, isothiocyanate, amine, alkyl halide, alkylmercurial halide, aldehyde, ketone, carboxylic acid halide, α-halocarbonyl, α,β-unsaturated carbonyl, haloformate ester, carboxylic acid, carboxylic ester, carboxylic anhydride, O-acyl isourea, hydrazide, maleimide, imidate ester, sulfonate ester, sulfonyl halide, α,β-unsaturated sulfone, aminooxy, semicarbazide, or β-aminothiol. In another embodiment, the valency platform molecule comprises at least 3 aminooxy groups and/or at least 3 carbamate groups.

In general, these platforms are made by standard chemical synthesis techniques. PEG must be derivatized and made multivalent, which is accomplished using standard techniques. Some substances suitable for conjugate synthesis, such as PEG, albumin, and IgG are available commercially.

For purposes of this invention, the valency platform molecules have a minimum valency of at least two, preferably at least four, preferably at least six, more preferably at least eight, preferably at least 10, preferably at least 12. As an upper limit, valency is generally less than 128, preferably less than 64, preferably less than 35, preferably less than 30, preferably less than 25, preferably less than 24, preferably less than 20, although the upper limit may exceed 128. Conjugates may also have valency of ranges of any of the lower limits of 2, 4, 6, 8, 10, 12, 16, with any of the upper limits of 128, 64, 35, 30, 25, 24, 20.

In some embodiments, the valency platform molecule comprises a carbamate linkage, i.e., —O—C(═O)—N<). Such platforms are described in a co-owned patent application entitled “Valency Platform Molecules Comprising Carbamate Linkages” U.S. Ser. No. 60/111,641 (U.S. Ser. No. 09/457,607 and PCT App. No. PCT/US99/29339), hereby incorporated by reference.

In other embodiments, valency platforms may be used which, when conjugated, provide an average valency (i.e., these platforms are not precisely chemically defined in terms of their valency). Examples of such platforms are polymers such as linear PEG; branched PEG; star PEG; polyamino acids; polylysine; proteins; amino-functionalized soluble polymers.

In some embodiments, the conjugates include branched, linear, block, and star polymers and copolymers, for example those comprising polyoxyalkylene moieties, such as polyoxyethylene molecules, and in particular polyethylene glycols. The polyethylene glycols preferably have a molecular weight less than about 10,000 daltons. In one embodiment, polymers with low polydispersity may be used. For example, polyoxypropylene and polyoxyethylene polymers and copolymers, including polyethylene glycols may be modified to include aminooxy groups, wherein the polymers have a low polydispersity, for example, less than 1.5, or less than 1.2 or optionally less than 1.1 or 1.07. Preferably, the polymers comprise at least 3 aminooxy groups, or at least 4, 5, 6, 7, 8, or more.

Conjugation of dsDNA Epitope(s) with Valency Platform Molecules

Conjugation of a biological or synthetic molecule to the chemically-defined platform molecule may be effected in any number of ways, typically involving one or more crosslinking agents and functional groups on the biological or synthetic molecule and valency platform molecule. Examples of standard chemistry which may be used for conjugation include, but are not limited to: 1) thiol substitution; 2) thiol Michael addition; 3) amino alkylation (reductive alkylation of amino groups); 4) disulfide bond formation; 5) acylation of amines.

The synthetic polynucleotide duplexes that are coupled to the valency platform molecule are composed of at least about 20 by and preferably 20-50 bp. Polynucleotides described herein are deoxyribonucleotides unless otherwise indicated and are set forth in 5′ to 3′ orientation. Preferably the duplexes are substantially homogeneous in length; that is, the variation in length in the population will not normally exceed about ±20%, preferably ±10%, of the average duplex length in base pairs. They are also preferably substantially homogeneous in nucleotide composition; that is, their base composition and sequence will not vary from duplex to duplex more than about 10%. Most preferably they are entirely homogeneous in nucleotide composition from duplex to duplex.

Based on circular dichroic (CD) spectra interpretation, duplexes that are useful in the invention assume a B-DNA type helical structure. It should be understood that it is not intended that the invention be limited by this belief and that the duplexes may, upon more conclusive analysis assume Z-DNA and/or A-DNA type helical structures.

These polynucleotide duplexes may be synthesized from native DNA or synthesized by chemical or recombinant techniques. Naturally occurring or recombinantly produced dsDNA of longer length may be digested (e.g., enzymatically, chemically and/or by mechanical shearing) and fractionated (e.g., by agarose gel or Sephadex™ column) to obtain polynucleotides of the desired length.

Alternatively, pairs of complementary single-stranded polynucleotide chains up to about 70 bases in length are readily prepared using commercially available DNA synthesizers and then annealed to form duplexes by conventional procedures. Synthetic dsDNA of longer length may be obtained by enzymatic extension (5′-phosphorylation followed by ligation) of the chemically produced shorter chains.

The polynucleotides may also be made by molecular cloning. For instance, polynucleotides of desired length and sequence are synthesized as above. These polynucleotides may be designed to have appropriate termini for ligation into specific restriction sites. Multiple iterations of these oligomers may be ligated in tandem to provide for multicopy replication. The resulting construct is inserted into a standard cloning vector and the vector is introduced into a suitable microorganism/cell by transformation. Transformants are identified by standard markers and are grown under conditions that favor DNA replication. The polynucleotides may be isolated from the other DNA of the cell/microorganism by treatment with restriction enzymes and conventional size fractionation (e.g., agarose gel, Sephadex™ column).

Alternatively, the polynucleotides may be replicated by the polymerase chain reaction (PCR) technology. Saiki et al (1985) Science 230:1350-1354; Saiki et al. (1988) Science 239:487-491; Sambrook et al. (1989) p 14.1-14.35.

The polynucleotides are conjugated to the chemically-defined valency platform molecule in a manner that preserves their antibody binding activity. This is done, for example, by conjugating the polynucleotide to the valency platform molecule at a predetermined site on the polynucleotide chain such that the polynucleotide forms a pendant chain of at least about 20 base pairs measured from the conjugating site to the free (unattached) end of the chain.

In one embodiment, the polynucleotide duplexes are substantially homogenous in length and one strand of the duplex is conjugated to the valency platform molecule either directly or via a linker molecule. Synthetic polynucleotides may be coupled to a linker molecule before being conjugated to a valency platform molecule. Usually the linker containing strand of the duplex is coupled at or proximate (i.e., within about 5 base pairs) to one of its ends such that each strand forms a pendant chain of at least about 20 base pairs measured from the site of attachment of the strand to the linker molecule. The second strand is then annealed to the first strand to form a duplex. Thus, a conjugate within the present invention may be generally described by the following formula: [(PN)ₙ-linker]ₘ-valency platform molecule wherein PN=a double-stranded polynucleotide with “n” nucleotides, wherein n=at least about 20 and m=2-8.

In one embodiment, the polynucleotides of the conjugates are coupled to a linker molecule at or proximate one of their ends. The linker molecule is then coupled to the chemically-defined valency platform molecule. As described in U.S. Pat. No. 5,552,391 and incorporated herein by reference, exemplary of suitable linker molecules within the present invention are 6 carbon thiols such as HAD, a thio-6 carbon chain phosphate, and HADₚ S, a thio-6 carbon chain phosphorothioate. Chemically-defined valency platform molecules within the present invention are formed, for example, by reacting amino modified-PEG with 3,5-bis-(iodoacetamido)benzoyl chloride (hereinafter “IA-DABA”); 3-carboxypropionamide-N,N-bis-[(6′-N′-carbobenzyloxyaminohexyl)acetamide] 4″-nitrophenyl ester (hereinafter “BAHA”); 3-carboxypropionamide-N,N-bis-[(8′-N′-carbobenzyloxyamino-3′,6′-dioxaoctyl)acetamide] 4″-nitrophenyl ester (hereinafter “BAHAₒₓ”); or by reacting PEG-bis-chloroformate with N,N-di(2-[6′-N′-carbobenzyloxyaminohexanoamido]ethyl)amine (hereinafter “AHAB”) to form chemically-defined valency platform molecules.

For example, a defined double-stranded polynucleotide (PN) can be conjugated to a valency platform molecule by first providing a single chain consisting of approximately 20 alternating cytosine (C) and adenosine (A) nucleotides. Four CA chains may then be covalently conjugated through linkers such as HAD to four reactive sites on a derivatized platform molecule such as triethylene glycol. The valency platform molecule is synthesized to include groups such as bromoacetyl. During the conjugation, a leaving group is displaced by sulfur. A second single nucleotide chain consisting of approximately 20 alternating thymidine (T) and guanosine (G) nucleotides can then be annealed to the CA strand to form a double-stranded PN conjugate of the formula, [(PN)₂₀-linker]₄-valency platform molecule.

Alternatively, in another embodiment, the polynucleotide may be coupled to the derivatized valency platform molecule at the 3′ end of the polynucleotide via a morpholino bridge formed by condensing an oxidized 3′ terminal ribose on one of the strands of the polynucleotide with a free amino group on the derivatized platform molecule and then subjecting the adduct to reducing conditions to form the morpholino linkage, as described in U.S. Pat. No. 5,553,391. Such coupling requires the derivatized platform molecule to have at least an equal number of amino groups as the number of polynucleotide duplexes to be bound to the platform molecule. The synthesis of such a conjugate is carried out in two steps. The first step is coupling one strand of the polynucleotide duplex to the derivatized platform molecule via a condensation/reduction reaction. The oxidized 3′ terminal ribose is formed on the single polynucleotide strand by treating the strand with periodate to convert the 3′ terminal ribose group to an oxidized ribose group. The single-stranded polynucleotide is then added slowly to an aqueous solution of the derivatized platform molecule with a pH of about 6.0 to 8.0 at 2-8° C., generally with a reducing agent (such as sodium borohydride).

The molar ratio of polynucleotide to platform molecule in all the conjugation strategies will normally be in the range of about 2:1 to about 30:1, usually about 2:1 to about 8:1 and preferably about 4:1 to 6:1. In this regard, it is preferable that the conjugate not have an excessively large molecular weight as large molecules, particularly those with repeating units, of m.w.>200,000 may be T-independent immunogens. See Dintzis et al. (1983) J. Immunol. 131:2196 and Dintzis et al. (1989) J. Immunol. 143:1239. During or after the condensation reaction (normally a reaction time of 24 to 48 hr), a strong reducing agent, such as sodium cyanoborohydride, is added to form the morpholino group. The complementary strand of the duplex is then added to the conjugate and the mixture is heated and slowly cooled to cause the strands to anneal. The conjugate may be purified by gel permeation chromatography.

An alternative to the ribose strategy is forming aldehyde functionalities on the polynucleotides and using those functionalities to couple the polynucleotide to the platform molecule via reactive functional groups thereon. Advantage may be taken of the fact that gem vicinal diols, attached to the 3′ or 5′ end of the polynucleotide, may be oxidized with sodium periodate to yield aldehydes which can condense with functional amino groups of the platform molecule. When the diols are in a ring system, e.g., a five-membered ring, the resulting condensation product is a heterocyclic ring containing nitrogen, e.g., a six-membered morpholino or piperidino ring. The imino-condensation product is stabilized by reduction with a suitable reducing agent; e.g., sodium borohydride or sodium cyanoborohydride. When the diol is acyclic, the resulting oxidation product contains just one aldehyde and the condensation product is a secondary amine.

Another procedure involves introducing alkylamino or alkylsulfhydryl moieties into either the 3′ or 5′ ends of the polynucleotide by appropriate nucleotide chemistry, e.g., phosphoramidite chemistry. The nucleophilic groups may then be used to react with a large excess of homobifunctional cross-linking reagent, e.g., dimethyl suberimidate, in the case of alkylamine derivatives, or an excess of heterobifunctional cross-linking reagent, e.g., m-maleimidobenzoyl-N-hydroxysuccinimide ester (MBS) or succinimidyl(4-iodoacetyl)aminobenzoate (SIAB), for the alkylsulfhydryl derivatives. Once excess cross-linker is removed, the polynucleotide derivatives are reacted with amino groups on the platform molecule. Alternatively, the sulfhydryl group may be reacted with an electrophilic center on the platform, such as a maleimide or α-haloacetyl group or other appropriate Michael acceptor.

Still another strategy employs modified nucleosides. Suitable deoxynucleoside derivatives can be incorporated, by standard DNA synthetic chemistry, at desired positions in the polynucleotide, preferably on the 5′ or 3′ ends. These nucleoside derivatives may then react specifically and directly with alkylamino groups on the platform molecule. Alternatively, side reactions seen with the above-described dialdehyde chemistry, such as amine catalyzed beta-elimination, can be circumvented by employing appropriate nucleoside derivatives as the 3′ terminus of the chain to be attached. An example of this is 5′ methylene extension of ribose; i.e., a 5′ (2-hydroxyethyl)-group instead of a 5′ hydroxymethyl group. An alternative would be to use a phosphonate or phosphinate linkage for the 3′ terminal dinucleotide of the polynucleotide to be attached to the platform molecule.

A description of the synthesis of the conjugate LJP 394, a tetravalent conjugate, is described in Jones et al. (1995) and in U.S. Pat. No. 5,552,391, which are hereby incorporated by reference. LJP 394 comprises four 20-mer oligonucleotides consisting of alternating C and A nucleotides, (CA)₁₀, attached to a platform and annealed with complementary 20-mer oligonucleotides consisting of alternating G and T nucleotides, (GT)₁₀, oligonucleotide. The valency platform molecule used in LJP 394 is shown immediately below.

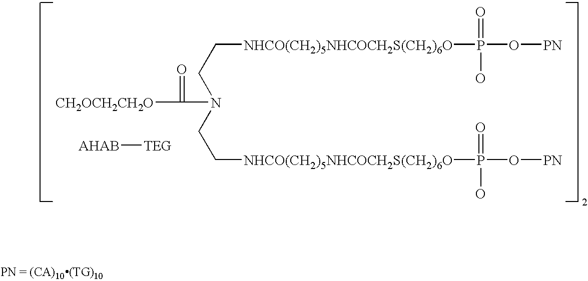

### Kits

The invention also provides kits for use in the instant methods. Kits of the invention include one or more containers comprising an epitope presenting conjugate (i.e., a conjugate comprising a non-immunogenic valency platform molecule and two or more double stranded DNA epitopes, preferably polynucleotides) and instructions for use in accordance with the methods of the invention. Accordingly, these instructions comprise a description of selecting an individual suitable for treatment based on identifying whether that individual has SLE and significant renal impairment (as indicated by any clinical indicia described herein and/or known in the art), and preferably also further describe administration of the conjugate for treatment of SLE and/or lupus nephritis. In some embodiments, the instructions comprise description of administering a conjugate to an individual having lupus nephritis who has significantly impaired renal function (which may also describe one or more criteria for determining whether an individual having, or suspected of having lupus nephritis has significant renal impairment). In some embodiments, the kits further comprise one or more compositions for measuring level of renal function in an individual.

In some embodiments, the kits may also contain supplies and instructions for measuring antibody affinities for use in the methods described herein, particularly affinity for an epitope which binds to anti-dsDNA antibodies. Accordingly, the kits of such embodiments contain (i.e., comprise) one or more dsDNA epitopes, preferably polynucleotides (preferably, double stranded (ds) DNA molecules) comprising an epitope which binds to an anti-dsDNA antibody from an individual (and the epitope-containing polynucleotide binds to an anti-dsDNA antibody from an individual). Accordingly, the kits comprise a molecule or moiety comprising a dsDNA epitope, such as any described herein. In one embodiment, the kit comprises a polynucleotide with (comprising) the sequence (or, alternatively, consisting essentially of or consisting of the sequence) 5′-GTGTGTGTGTGTGTGTGTGT-3′(SEQ ID NO:1). In certain embodiments the dsDNA epitopes are not part of a conjugate with a non-immunogenic valency platform molecule. In other embodiments, the kits comprise the conjugates described herein, with instructions for using the conjugate to detect affinity of an individual's anti-dsDNA antibodies for the conjugate. Preferably, the conjugate is LJP 394.

In those embodiments containing materials and instructions for measurement of antibody affinity, such materials may be used, for example, to test an individual to determine if the individual is suitable or unsuitable for treatment with the conjugate(s), as well as for monitoring purposes. The affinity testing materials may also be used in determining affinity cut-off values (i.e., affinity values which correlate with clinical results).

The kits of this invention are in suitable packaging. Suitable packaging for epitope presenting conjugates includes, but is not limited to, vials, bottles, jars, flexible packaging (e.g., sealed Mylar or plastic bags), and the like.

Kits may optionally provide additional components such as, buffers and instructions for determining affinity or binding to anti-dsDNA antibody, such as capture reagents, developing reagents, labels, reacting surfaces, means for detection, control samples, and interpretive information. The instructions relating to measurement of antibody affinity may be for any measurement of antibody affinity, including, but not limited to, those assays described herein. Accordingly, in some embodiments, the instructions are for determining affinity using surface plasmon resonance. In other embodiments, the instruction are for determining affinity using direct binding assays and/or Farr assays. In some embodiments, reagents described above are supplied such that multiple measurements may be made, such as allowing for measurements in the same individual over time or multiple individuals.

In those embodiments comprising materials for testing antibody affinity, the dsDNA epitope(s) of the kit, preferably a polynucleotide(s) of the kit (whether in free form or attached to a conjugate or other matrix), generally contains, or alternatively consists of, the epitope that will be or is used in treatment, or has been demonstrated to have about the same affinity for an individual's anti-dsDNA antibodies as the epitope(s) that will be used in treatment. In other embodiments, the kits comprising a dsDNA epitope whose affinity for anti-dsDNA antibodies mimics or alternatively can be correlated to that of the dsDNA epitope to be used in treatment, such as 5′-GTGTGTGTGTGTGTGTGTGT-3′(SEQ ID NO:1). These dsDNA epitopes can be used as “proxies” for the dsDNA epitope to be used in treatment, such as LJP 394, in assessing antibody affinity for the methods described herein.

Embodiments including materials for testing antibody affinity may comprise any appropriate means for detecting binding of the antibodies, such as a labeled anti-human antibody, when the presence of human anti-dsDNA antibodies is tested, wherein the label may be an enzyme, fluorophore, chemiluminescent material radioisotope or coenzyme. Generally, the label used will be an enzyme. Accordingly, in some embodiments, the kit(s) of the invention further comprises a label. In some embodiments, the polynucleotide in the kit(s) is conjugated to biotin. In a preferred embodiment, the dsDNA epitope (such as a polynucleotide, for example, double stranded DNA) is biotinylated. Biotinylation may also be accomplished using commercially available reagents (i.e., Pharmacia; Uppsala, Sweden). In another preferred embodiment, the biotinylated dsDNA epitope comprises, consists essentially or, or consists of is 5′-GTGTGTGTGTGTGTGTGTGT-3′(SEQ ID NO:1).

In other embodiments, the invention provides a kit comprising (a) an epitope presenting conjugate as described herein, such as LJP 394; and (b) a polynucleotide (or other dsDNA epitope) used in the conjugate, or, alternatively, a polynucleotide comprising the polynucleotide used in the conjugate (or a molecule or moiety comprising the epitope to be used in the conjugate). These kits also contain the instructions for practicing a method(s) of the invention, as described above. When used for affinity measurements, the conjugate and/or polynucleotide may be biotinylated. In some embodiments, the kit contains instructions for administering the conjugate to an individual as well as instructions for using the conjugate and/or the polynucleotide (including a polynucleotide comprising the polynucleotide used in the conjugate) for detecting affinity for an antibody in an individual which binds to dsDNA as described herein. As discussed herein, a combination of a conjugate to be used for treatment and a molecule comprising a dsDNA epitope, the binding activity or affinity of which mimics, or can be correlated with, the epitope of the conjugates is used in the kits.

The following Examples are provided to illustrate but not limit the invention.

### Examples

### Example 1

### Treatment of SLE Patients Having Significantly Impaired Renal Function with LJP 394

230 SLE patients were enrolled in a double-blind, placebo-controlled trial of LJP 394 (abetimus sodium). The study was a multicenter, double-blind, randomized, parallel group trial comparing intravenously administered LJP 394 to placebo during induction (100 mg weekly) and maintenance periods (50 mg weekly) in patients with a history of lupus renal disease. LJP 394 is formulated as a sterile, colorless liquid in an isotonic phosphate-buffered saline solution for intravenous (IV) administration. Each 1 mL of solution contains 50 mg of LJP 394, 1.9 mg Na2HPO4*7H20, 0.30 mg NH₂PO₄*H₂0, and 5.8 or 8.0 mg NaCl in water for Injection, USP (pH 6.8-8.0). The formulation contains no preservatives.

Prospective patients were observed for four to six weeks prior to randomization to ensure that entrance criteria were met prior to randomization. The initial protocol provided LJP 394 or placebo at 100 mg/week for 52 weeks with a six month follow up period. The protocol was later amended to a 75 week treatment period consisting of a 15 week induction period and a 60 week maintenance period consisting of 15 weekly doses of LJP 394 or placebo, followed by alternating eight week drug holidays and 12 weekly treatments with 50 mg LJP 394 or placebo for a total of 75 weeks. All patients in the original protocol were transferred into the amended protocol prior to the end of the first dosing cycle of the maintenance period.

A protocol-defined renal flare was reached if one or more of the following endpoints was met and the flare was attributed to SLE by the treating physician and the study's medical monitor: (a) reproducible increase in 24 hour urine protein to greater than 1000 mg per 24 hours if the screening value was less than 200 mg per 24 hours, to greater than 2000 mg per 24 hours if the screening value was 200-1000 mg per 24 hours, or to more than two fold the screening value if it was greater than 1000 mg per 24 hours; (b) a reproducible increase in serum creatinine of >20% or at least 0.3 mg/dL, which ever was greater, accompanied by proteinuria (>1000 mg per 24 hours), hematuria (>four red blood cells per high power field (RBCs/HPF)) and/or red cell casts; or (c) new reproducible hematuria (>11-20 RBCs/HPF) or a reproducible two grade increase in hematuria compared to baseline with >25% dysmorphic blood cells (glomerular in origin), exclusive of menses, and either an 800 mg increase in 24 hour protein or new red cell casts.

Baseline for dsDNA was calculated as the mean of the last two screening measurements. Baseline for all other laboratory values was the measurement taken immediately prior to the first administration of study drug.

Therapeutic intervention with high doses of corticosteroids and/or cyclophosphamide (HDCC) was left to the investigator's discretion and summarized at study closure using the following criteria: any exposure to cyclophosphamide; systemic prednisone (or prednisone equivalent) increase ≧15 mg per day over baseline dose to greater than 20 mg per day for more than two days or a dose of prednisone (or prednisone equivalent) that exceeded 200 mg in a single day. Topical, intra-articular, or intra-ocular usage was excluded.

The comparison of continuous variables was performed using analysis of variance. The Fisher's exact test was used for the comparison of incidence rates and all other categorical comparisons. No adjustments were made to p-values for multiple comparisons.

All time-to-event comparisons were performed using the Kaplan-Meier product limit method and the log rank test. When comparing time-to-event variables for the entire study period, all patients were included in the analysis until time of event (renal flare or HDCC) or their last exposure to drug. Only the first renal flare and the first exposure to HDCC were used in the time to event and incidence analysis.

Two hundred and thirty patients were randomized to receive study drug (116 received LJP 394, 114 received placebo). Pretreatment samples for determination of affinity were available for 213 patients. The trial was prematurely terminated after an interim analysis established that the trial was unlikely to reach statistical significance for time to renal flare in the ITT (intent to treat) population.

Twenty seven patients (17 receiving LJP 394, 10 receiving placebo) were enrolled exhibiting significantly impaired renal function (i.e., with baseline serum creatinine levels between 1.5 mg/dL and 2.5 mg/dL (the upper limit for inclusion). In these patients, renal flare was observed in 3/17 (18%) of LJP 394-treated patients and 6/10 (60%) placebo patients. LJP 394-treated patients had a significantly longer time to renal flare than patients treated with placebo (p=0.003). Eleven of 17 (65%) of LJP 394 patients and all of the placebo patients had high affinity antibodies to LJP 394 prior to treatment. Of the patients with high affinity antibodies (Kd′ less than or equal to 0.8 mg IgG/ml), there were no renal flares in the LJP 394-treated group, compared to 6/10 (60%) of the placebo group (p=0.004).

Patients with significantly impaired renal function at baseline appeared to fare worse than the overall study population. Six of ten (60%) patients in the placebo group developed renal flares versus 23/116 (20%) in the ITT placebo group. LJP 394-treated patients that did not have high affinity antibodies to LJP 394 fared poorly (same as the placebo population, in which 6 of 10 developed renal flares), with three of six (50%) developing renal flares, as compared to 19/116 (16%) of the LJP 394 ITT population, and 7/92 (8%) of the LJP 394 treated patients having high affinity antibodies. However, there were no renal flares amongst those patients having significantly impaired renal function and high affinity antibodies to LJP 394.

### Example 2

### Inhibition of Binding of Anti-dsDNA Antibodies to DNA by LJP 394

After determining the presence of anti-dsDNA antibodies in patients using a Farr assay, a competitive Farr assay was used to measure the affinity of anti-dsDNA antibodies found in sera from patients with SLE to LJP 394. In addition, the assay was used to measure the affinity of anti-dsDNA antibodies found in sera from three animals models of SLE (BXSB mice, NZB×NZW F₁ mice, and MRL/lpr mice).

The Farr assay used ¹²⁵I-labeled recombinant dsDNA (Diagnostic Products Corporation, Los Angeles, Calif.) that was combined with the anti-dsDNA antibodies found in sera from patients with SLE or from the mouse models of SLE. Anti-dsDNA antibodies were obtained from serum samples of donors with SLE collected through a volunteer donor program. Blood samples were drawn, serum harvested, aliquots made, labeled, and stored frozen at −70° C. until used. In this assay, 25 μL of patient's serum was added to 75 μL of Tris buffer (50 mM Tris, 150 mM NaCl pH 7.5, 10% normal rabbit serum), then 100 μL of ¹²⁵I-labeled recombinant dsDNA was added, mixed and incubated at 37° C. for one hour. Similar samples containing known amounts of anti-dsDNA antibodies (calibrators) were prepared and incubated at the same time. 500 μL of 70% saturated ammonium sulfate was added to each tube, mixed, and then centrifuged at 800×g for 15 minutes to precipitate the antibodies in solution. The supernatant was decanted and the amount of radioactivity in the precipitated product was determined by counting the radioactivity in a gamma counter. The amount of radioactivity in the precipitant is proportional to the amount of anti-dsDNA antibodies that bound to ¹²⁵I-labeled recombinant dsDNA. Calibrators with known amounts of anti-dsDNA antibodies were used to generate a standard curve from which the amount of dsDNA binding by anti-dsDNA antibodies could be calculated.

Serum samples from 58 patients were assayed for the presence of antibodies to dsDNA using the Farr assay described above. Forty-two of these samples had sufficient levels of antibody 20% binding) to use in the LJP 394 inhibition assay.

LJP 394 was tested for its ability to inhibit binding of anti-dsDNA antibodies to ¹²⁵I-labeled recombinant dsDNA by a competitive Farr assay. Calf thymus DNA (ctDNA) was also used in the inhibition assay as another source of dsDNA. Calf thymus dsDNA was prepared by dissolving calf thymus DNA in nuclease-S1 buffer (0.2 M NaCl, 50 mM sodium acetate pH 4.5, 1 mM ZnSO₄ and 0.5% glycerol) and 100,000 units of S-1 nuclease and incubating for one hour at 37° C. The dsDNA was extracted from this mixture by adding an equal volume of phenol-chloroform, mixing, centrifuging, and harvesting the aqueous layer. The dsDNA was then precipitated by adding 2 volumes of EtOH, mixing, and centrifuging. The pellet was harvested, dried under vacuum and dissolved in water to approximately 10 mg/mL. The final concentration of the ctDNA preparation was determined spectrophotometrically assuming an extinction coefficient of 33 μg per 1 OD unit at 260 nM.

Each serum sample that gave 20% binding was tested in the inhibition assay. Briefly, 25 μL of patient's serum was added to 75 μL of Tris buffer (50 mM Tris, 150 mM NaCl pH 7.0, 10% normal rabbit serum) containing various concentrations of inhibitor (either calf thymus dsDNA or LJP 394), then 100 μL of ¹²⁵I-labeled recombinant dsDNA was added, mixed and incubated at 37° C. for one hour. 500 μL of 70% saturated ammonium sulfate was added to each tube, mixed and then centrifuged at 800×g for 15 minutes. The supernatant was decanted and the amount of radioactivity in the precipitated product was determined by counting the radioactivity in a gamma counter. Extent of inhibition was calculated by the following formula: {[(cpm patient's serum without inhibitor−cpm without patient's serum, no inhibitor)−(cpm patient's serum with inhibitor−cpm without patient's serum, no inhibitor)] divided by (cpm patient's serum without inhibitor−cpm without patient's serum, no inhibitor)} all times 100.

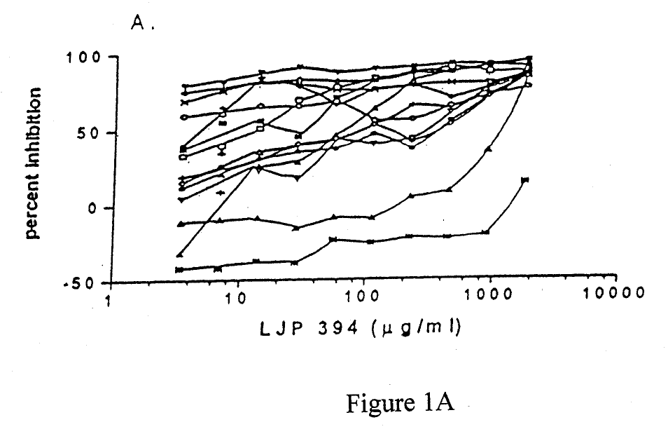

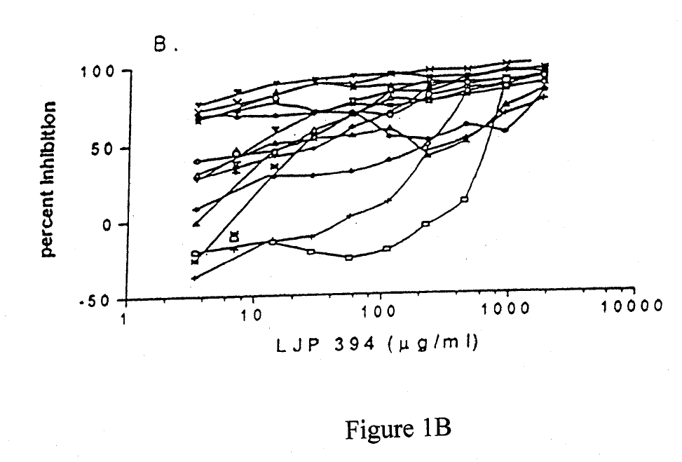

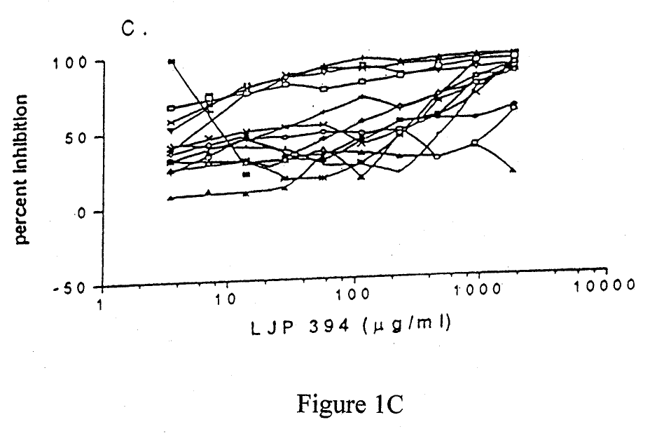

FIGS. 1A-C illustrate the ability of LJP 394 to inhibit the binding of autoantibodies from a representative populations of patients with SLE. Overall, LJP 394 was capable of inhibiting binding of the autoantibodies to dsDNA in 42 out of 42 patients with SLE. The inhibition curves for LJP 394 and calf thymus dsDNA were parallel, suggesting that the antigenic determinants being recognized by the SLE sera were identical on both the calf thymus dsDNA and LJP 394.

The ability of LJP 394 to inhibit the binding of anti-DNA antibodies in a mouse models of SLE was also tested. Competitive inhibition assays with calf thymus dsDNA and LJP 394 were performed as described above and the results are shown in Table 1. The 50% inhibition ratios (IC₅₀ LJP 394/IC₅₀ ctDNA) were lowest for human anti-dsDNA antibodies (from SLE sera), compared to the mouse antibodies. LJP 394 showed high affinity for human antibodies and the NZB×NZW F1 mouse strain.

TABLE 1
Competitive Inhibition of Binding of Anti-dsDNA
Antibodies by ctDNA and LJP 394

| | | | | IC₅₀ LJP |
|---|---|---|---|---|
| No. of | IC₅₀, μg/mL (mean ± SD) | | | 394/ctDNA |
| sera | Source of sera | ctDNA | LJP 394 | ratio |
| 3 | MRL | 0.356 ± 0.455 | 200 ± 42 | 562 |
| | (lpr/lpr)(mouse) | | | |
| 3 | NZBxNZWF₁ | 0.021 ± 0.011 | 5.5 ± 0.7 | 258 |
| | (mouse) | | | |
| 5 | BXSB (mouse) | 0.028 ± 0.000 | 215 ± 144 | 7679 |
| 42 | Human SLE | 1.88 ± 0.920 | 46 ± 16 | 24 |

### Example 3

### Determination of Titer-Weighted Average Affinity of Antibodies for Conjugate by a Surface Plasmon Resonance Assay

An assay using surface plasmon resonance is used to directly measure a titer-weighted average affinity of antibodies from SLE patients for the conjugate LJP 394. Surface plasmon resonance is used to quantify the fractional saturation of antigen with antibody. This assay was adapted so that it measures the titer weighted average affinity of the IgG population of LJP 394.

### Materials and Methods

Reagents. Streptavidin CM5 chips, HBS buffer (0.01 M HEPES pH 7.4, 0.15 M NaCl, 3 mM EDTA and 0.005% (v/v) surfactant P20) are obtained from BIACORE AB.

LJP 394 is composed of four 20-mer dsDNA epitopes that are covalently attached to a triethyleneglycol-based platform by a thiol linkage. The DNA epitope is composed of 5′-(CA)₁₀-3′ strands annealed to complementary GT strands, with biotin attached at the free 5′ ends of the GT strand. Biotin is incorporated by using Biodite biotin amidite (Pharmacia) in the final coupling of the (GT)₁₀ strand. LJP 394 is prepared essentially as described in Jones et al. (1995) except that this biotin-modified (GT)₁₀ strand is used in the annealing step. In some experiments, only the dsDNA epitope is immobilized on the streptavidin chip. The epitope is prepared by annealing 5′-(CA)₁₀-3′ to 5′-biotin-(TG)₁₀-3′ and purifying the dsDNA by HPLC.

Plasma samples are collected from SLE patients, and total IgG fraction is isolated from the plasma by combining 100 μL of plasma with 100 μL of IgG binding buffer (Pierce Chemical Co.; Rockford, Ill.) and mixing with Immunopure Plus® protein G agarose beads (Pierce Chemical Co.) according to manufacturer recommendations. Elution of IgG from the beads is accomplished by following the acid elution/neutralization protocol of Pierce Chemical Co., and 300 μL of acid eluted IgG is neutralized with 100 μL of 1 M NaPO₄, pH 7.5. These purified IgG samples are then used in the titration experiments. Total IgG concentrations are determined with the Bradford assay (Biorad; Hercules, Calif.).

Surface Plasmon Resonance. All measurements are performed using a BIACORE® 2000 instrument at 25° C. with a flow rate of 10 μl/minute. LJP 394 is attached to the streptavidin CM5 chip through its 5′ biotin group by flowing a 50 μg/mL solution of LJP 394 in HBS+0.3 M NaCl over the chip for 20 minutes at 5 μL/minute. The chip is preconditioned prior to titration with 3×1 minute pulses of regeneration buffer (1M NaCl and 50 mM NaOH). When the dsDNA epitope of LJP 394 is used for immobilization, the biotinylated epitope is flowed over the chip at a concentration of 10 μg/mL using similar conditions as employed for the biotinylated LJP 394 epitope.

Antibody titrations of the dsDNA (LJP 394) chip are performed with serial 1:2 dilutions of purified IgG in HBS. Sample is injected for 7 minutes, which is adequate association time for a significant approach to the response plateau, and is followed by a 4 minute dissociation period where HBS is flowed over the chip, then a 30 second regeneration is performed with 1 M NaCl, 50 mM NaOH. These regeneration conditions appear to cause denaturation of at least some of the DNA on the chip for the oligomer used in LJP 394.

Analysis. Response plateau values (R_{eq}) are obtained by a nonlinear least squares fit of the association curves to equation 1, after subtraction of a background curve for an empty flow cell, to account for bulk response/buffer effect, and using the manufacturers software (BiaEvaluation version 2.2, Uppsala, Sweden)

Rₜ=R_{eq}(1−e^{-ks(t-t0)})+R₀ (equation 1)

where Rₜ is the measured response at time t, R_{eq} is the equilibrium plateau response, t is time, t₀ is initial time, kₛ is an apparent association constant (kₛ=kₐC+k_{dis}, where kₐ is the association constant, C is the analyte concentration and k_{dis} is the dissociation constant), and R₀ is a response offset. These response plateaus are plotted versus the concentration of total IgG, and fitted to equation 2 to obtain values for Rₘₐₓ and K_{d}*.

$${R}_{eq} = \frac{{R}_{max} {A}_{T}}{{K}_{d}^{*} + {A}_{T}} \qquad ( equation 2 )$$

where A_{T} is the total antibody (IgG) concentration, Rₘₐₓ is the maximum response plateau and K_{d}* is an apparent dissociation constant. K_{d}* is the same as <K_{d}′> in equation 3 (below), the titer-weighted-average (TWA) dissociation constant. The derivation of K_{d}′ was performed as described in Sem et al. ((1999) Arch. Biochem. Biophys. 372:62-68) and provides insight into the physical meaning of the K_{d}* constant in equation 2. This analysis pertains to the case of a polyclonal pool of n different antibody subpopulations, where B=LJP 394 and Aᵢ=antibody subpopulation i.

$$〈 {K}_{d}^{′} 〉 = \frac{{A}_{T}}{{\sum }_{i = 1}^{n} ( {A}_{i} / {K}_{i} )} = \frac{1}{{\sum }_{i = 1}^{n} ( {r}_{i} / {K}_{i} )} \qquad ( equation 3 )$$

where rᵢ (relative titer) is the fraction of total antibody present as form i, defined as rᵢ=Aᵢ/A_{T}. Thus, equation 3 is the general equation describing the observed dissociation constant for a polyclonal population of n different antibody subpopulations of relative titer (fractional presence) rᵢ and dissociation constant Kᵢ. This <K_{d}′> is the apparent K_{d} of equation 2, K_{d}*.

The measured apparent dissociation constant K_{d}′ reflects both inherent affinity of antibody subpopulation i for antigen, and relative titer of antibody subpopulation i (rᵢ). In general, 0<rᵢ<1, so K_{d}′>Kᵢ. That is, the factors that can cause K_{d}′ to decrease are an increase in affinity (Kᵢ decreases) and/or an increase in relative titer of antibody subpopulation i (rᵢ increases). In practice, in a polyclonal population of antibodies, there will be many different antibody subpopulations that bind, each with slightly different affinity.

The above analysis, and that further described in Sem et al. (1999), produces an apparent dissociation constant that is a reflection of the various affinities and titers of clonally related subpopulations of antibodies within a polyclonal pool. The apparent dissociation constant obtained as described is the titer-weighted-average (TWA) dissociation constant derived in equation 3, <K_{d}′>. The value of <K_{d}′> is dominated by antibody subpopulations that have the largest rᵢ (highest relative titer) and smallest Kᵢ (highest affinity) in combination. Any change in relative titers of subpopulations with a given affinity will change the apparent dissociation constant according to equation 3.

1. A method of reducing incidence of renal flares in a systemic lupus erythematosus patient having significantly impaired renal function, comprising administering to the patient an effective amount of a conjugate comprising a non-immunogenic valency platform molecule and two or more double stranded DNA (dsDNA) epitopes, wherein the double stranded DNA epitopes are polynucleotides.

2. The method of claim 1, wherein the polynucleotides are double stranded DNA.

3. The method of claim 2, wherein said polynucleotides comprise the sequence 5′-TGTGTGTGTGTGTGTGTGTG-3′ (SEQ ID NO:1).

4. The method of claim 3, wherein the platform molecule is 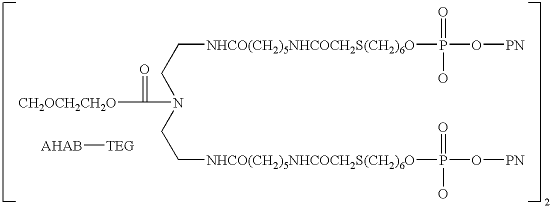
wherein PN is the polynucleotide.

5. The method of claim 1, wherein said patient has serum creatinine of greater than 1.5 milligrams per deciliter (mg/dL).

6. A method of treating systemic lupus erythematosus (SLE) in an individual, comprising selecting an individual having SLE and significantly impaired renal function, and administering to the individual an effective amount of a conjugate comprising a non-immunogenic valency platform molecule and two or more double stranded DNA (dsDNA) epitopes, wherein the dsDNA epitopes are polynucleotides.

7. The method of claim 6, wherein the polynucleotides are double stranded DNA.

8. The method of claim 7, wherein said polynucleotides comprise the sequence 5′-TGTGTGTGTGTGTGTGTGTG-3′ (SEQ ID NO:1).

9. The method of claim 8, wherein said polynucleotides consist essentially of the sequence 5′-TGTGTGTGTGTGTGTGTGTG-3′ (SEQ ID NO:1).

10. The method of claim 9, wherein the polynucleotide consists of the sequence 5′-TGTGTGTGTGTGTGTGTGTG-3′ (SEQ ID NO:1).

11. The method of claim 10, wherein the platform molecule is 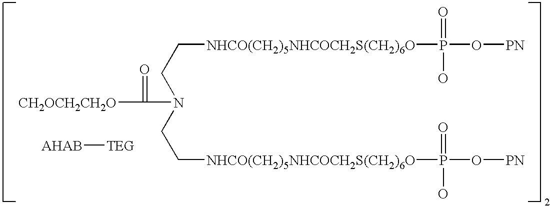
wherein PN is the polynucleotide.

12. The method of claim 1 or 6, wherein before or upon initiation of treatment the individual comprises antibodies having an apparent equilibrium dissociation constant (K_{D}′) for a polynucleotide of the conjugate of less than about 1.0 mg IgG per mL.

13. The method of claim 12, wherein the K_{D}′ is less than about 0.8.

14. The method of claim 12, wherein the K_{D}′ is less than about 0.5.

15. The method of claim 12, wherein the K_{D}′ is less than about 0.2.

16. The method of claim 1 or 6, wherein affinity of antibodies from the individual for a polynucleotide of the conjugate is assessed.

17. The method of claim 16, wherein said affinity is assessed prior to or upon initiation of treatment of the individual, thereby producing an initial affinity measurement.

18. The method of claim 17, wherein the initial affinity measurement is an apparent equilibrium dissociation constant (K_{D}′).

19. The method of claim 18, wherein the K_{D}′ is less than about 1.0 mg IgG per mL.

20. The method of claim 18, wherein the K_{D}′ is less than about 0.8.

21. The method of claim 18, wherein the K_{D}′ is less than about 0.5.

22. The method of claim 6, wherein the platform molecule is 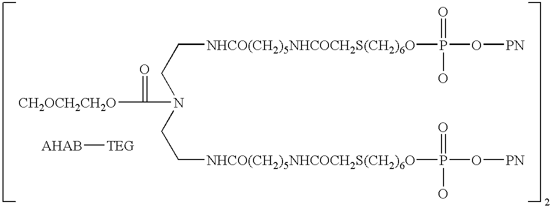
wherein PN is the polynucleotide.

23. The method of claim 6 wherein the conjugate is administered in an amount effective to reduce incidence of renal flares in the individual.

24. The method of claim 1 or 6, wherein a medication selected from the group consisting of corticosteroids and cyclophosphamide is also administered to the individual.

25. The method of claim 24 wherein the conjugate is administered in an amount effective to reduce the amount of a corticosteroid or cyclophosphamide administered to the individual.

26. The method of claim 1 or 6, wherein the individual is human.

27. A method of treating systemic lupus erythematosus (SLE) in an individual, comprising selecting an individual having (a) SLE, (b) significantly impaired renal function, and (c) antibodies with high affinity to a polynucleotide epitope a conjugate comprising a non-immunogenic valency platform molecule and two or more polynucleotides, said polynucleotides comprising at least one double stranded DNA epitope, and administering to the individual an effective amount of said conjugate.
